(19) United States
(12) Patent Application Publication
(10) Pub. No.: US 2011/0238022 A1
(43) Pub. Date: Sep. 29, 2011

(54) CORPOREAL DRAINAGE SYSTEM

(76) Inventors: Shayna Massi, Chicago, IL (US); Griffin Strole, Chicago, IL (US); Jim Kantola, Walikegon, IL (US); Tony Looper, Lake Zurich, IL (US); Monica Sanders, Oak Park, IL (US); Maria Lukas, Anitoch, IL (US); Steve Latham, Sun Prairie, WI (US); Jesse Darley, Madison, WI (US); Nick Reback, Madison, WI (US); Kent Kallsen, Jefferson, WI (US)

(21) Appl. No.: 12/730,925

(22) Filed: Mar. 24, 2010

(51) Int. Cl. A61M 1/00

(52) U.S. Cl. 604/319

(57) ABSTRACT

A system and method for collecting fluid from a patient is disclosed. The system includes two plates separated by restoring force providing members and straps allowing the system to be placed in expanded and compressed modes. The system is a single use system that prevents the re-use of the system once it has been drained of collected fluid. Methods for fabricating the system are also disclosed, including a method that bonds two plates together to create an assembled version of the system.

### BACKGROUND OF THE INVENTION

1. Field of the Invention

The present invention relates to medical drainage devices and particularly to medical drainage devices involving the application of negative pressure or vacuum. The present invention also relates to hand, spring, restoring force or otherly actuated medical drainage devices. One aspect of the present invention relates to a hand, spring, restoring force or otherly actuated medical drainage device for use in draining excess fluid from the body or for draining excess fluid from some other entity.

2. Background of the Related Art

Fluid extraction devices and fluid evacuators for patients suffering from symptoms associated with excess fluid buildup are known. Relatively common maladies that cause excess fluid build-up include pleural effusion or ascites, excess accumulation of fluid in the pleural or peritoneal cavity, respectively, among others. The practice of inserting a catheter into a patient with pleural effusion, ascites, or similar maladies, and drawing excess fluid through the catheter so that the fluid may be collected in a receptacle is well-established medical procedure. Among other benefits, the removal of excess fluid can increase a patient's comfort level and decrease the risk of infection. In addition, there are many other reasons that fluid extraction is necessary or beneficial, including that excess fluid may obstruct diagnostic probes or other medical analyses from being performed.

Typically, medical fluid evacuation employs a vacuum source, such as a vacuum bottle or pump, to draw fluid from the patient. A “vacuum bottle” is generally a reservoir, bottle or other receptacle which has been provided with a negative pressure and then sealed. Examples include the Pleurex device (U.S. Pat. No. 5,484,401). Vacuum pumps are common, despite the fact that they can be cumbersome and relatively restricting for active patients. Such pumps generally require an active power supply and, therefore, necessarily must be used within range of the power supply (e.g., within the limits of power cords or other conveyances). Active vacuum pumps having mobile power supplies, such as those supplied by batteries, known to be cumbersome and heavy, which can be restrictive for patients in a weakened condition. Vacuum bottles do not require external power supplies and, therefore, overcome at least some of the above-discussed disadvantages. However, maintaining a constant vacuum in such bottles, particularly over the course of a fluid extraction which may take hours is known to be rather difficult. Further, some vacuum bottles are not delivered to the patient or the caregiver in a pre-compressed or vacuum state. Therefore, the caregiver or patient must provide the vacuum, which can be awkward or difficult. In particular, evacuation of the bottles may require additional, cumbersome equipment such as vacuum pumps, etc. and/or the application of physical force.

Another alternative that has been developed uses reservoirs that provide a vacuum-drawing restoring force. Examples include reservoirs using springs or other restoring force providing members (see, e.g., U.S. Pat. No. 4,429,693, U.S. Pat. No. 4,161,179 and references discussed therein). However, many of these latter devices use clamps or other mechanical locking systems to prevent the accidental discharge of vacuum during shipping. Such clamps or other mechanical locking mechanisms can be difficult to operate by the users, particularly if the user is a patient suffering from a debilitating illness. Moreover, the locking mechanism and/or restoring force providing members can be so bulky as to limit the compressibility of the overall system. In general, it can be advantageous for the system to be as compressible as possible so that multiple systems or devices can be delivered to the patient or caregiver relatively easily and so the multiple systems or devices can be easily stored in the vicinity of the patient. It can also be advantageous for the device to be relatively light and portable. Many devices and systems using clamps, locking mechanisms and traditional restoring force providing members are rather heavy and difficult to move or maneuver.

Often, reservoirs or devices and systems for fluid extraction are not delivered in the compressed states which can be disadvantageous because the un-compressed devices or systems are often bulkier and more difficult to deliver and because the uncompressed devices or systems require evacuation by either the patient or the caregiver. The former increases delivery and storage costs. The latter can render the systems or devices difficult to use and implement. Since it can be difficult to manually compress spring-loaded or other devices, doing so is often too difficult or demanding for the patient.

Most currently available devices and system for medical fluid draining are not necessarily single-use systems and do not prevent either inadvertent or intentional re-use. Since the medical fluid extraction treatment can, and often is, administered by the patient, there is a temptation and a danger that such devices may be re-used. Re-using devices for medical fluid extraction poses a variety of potential problems, including, but not limited to, infection resulting from unsanitary conditions created by the accumulation of medical fluid. Even if the user empties the medical fluid draining system or device and believes the devices is fit for re-use, the user has likely not adequately sterilized or cleaned the device. Indeed, medical sterilization is often difficult and involves the use of complicated devices. In other words, medical grade sterilization is typically not easily performed by patients who often lack specialized medical knowledge or even a rudimentary understanding of such sterilization procedures and protocols. Further, patients suffering from debilitating illnesses may not be physically capable of performing a thorough sterilization of a complicated fluid extraction device. The same patients may have an increased susceptibility to the negative side effects of various cleaning products and chemicals used in sterilization. As a result, it is generally advantageous for medical fluid extraction devices to be disposed of after the first and only use in order to prevent attempts at re-use by the user. Yet, many currently available medical fluid extraction devices and systems, even if designed to be disposable, generally lack a mechanism for preventing re-use of the system.

In general, it is also relatively difficult to assemble complex devices that use springs or restoring force providing members and, yet, also maintain fluid and vacuum-tight seals. In particular, embedding the restoring force providing members in the device or system and creating a fluid and vacuum-tight seal is often difficult and expensive. The manufacture of such devices or systems is correspondingly slow and prone to error.

Therefore, there is a need in the art for a medical fluid extraction or draining device that provides a vacuum in a way that does not inconvenience, impair, restrict or require difficult maintenance from the user. There is also a need in the art for a medical fluid extraction or draining device that is relatively compressible, storable and transportable. Further, there is a need in the art for a medical fluid extraction or draining device that is configured such that it is not re-usable after a single use. In addition, there is a need in the art for a medical fluid extraction or draining device that is relatively cost effective and easy to manufacture.

### SUMMARY OF THE INVENTION

While the discussion of the aspects of the present invention that follows uses surgery for an illustrative purpose, it should be appreciated that the environment of the present invention is not limited to surgery. Aspects of the invention may be used in a variety of other environments. For example, aspects of the present invention may be used in fluid extraction relating to manufacturing, construction, assembly lines, handling and disposing of hazardous materials, underwater manipulations, handling high temperature materials, or any other environment where a user may need to extract fluid from an entity.

Aspects of the present invention may aid a user, for example, a surgeon or other such medical practitioner in extracting excess fluid from a patient. A restoring force providing member creates a vacuum in a system or device which draws fluid from a patient through a catheter or other device. The system or device may be provided to the user in a pre-compressed state so that the user may create or “pull” a vacuum by simply releasing the system. Additional aspects of the present invention provide a medical fluid extracting device or system that can be configured for only a single use such that emptying the device or system renders it inoperable.

One aspect of the present invention includes a corporal drainage system for draining fluid from a patient, the system including: two plates positioned opposite each other; at least one restoring force providing member positioned between the plates; a mechanism for securing the plates to one another in a compressed mode wherein the at least one restoring force providing member is compressed; a mechanism for releasing the two plates in an extended mode wherein the at least one restoring force providing member is extended; a seal between the two plates and an intake port, such that when the plates are in the extended mode, a vacuum is exerted on the intake port; and a conduit connecting the intake port to the patient such that the vacuum exerted on the intake port draws fluid from the patient.

In another aspect of the present invention, a corporal drainage system for draining fluid from a patient includes: two plates positioned opposite each other; a reservoir formed between the plates wherein the reservoir includes a flexible material that is divided into sections, a first section being bonded to one of the plates and a second section being bonded to the opposite plate; at least one restoring force providing member positioned between the plates; a mechanism for securing the plates to one another in a compressed mode wherein the at least one restoring force providing member is compressed; a mechanism for releasing the two plates in an extended mode wherein the at least one restoring force providing member is extended; a seal between the two plates and an intake port such that, when the plates are in the extended mode, a vacuum is exerted on the intake port, wherein the seal includes a seam bonding the first and second sections of the reservoir to one another; and a conduit connecting the intake port to the patient such that the vacuum exerted on the intake port draws fluid from the patient.

In yet another aspect of the invention, a method for fabricating a corporal drainage system for draining fluid from a patient includes: bonding a first film to a first plate and second film to a second plate wherein each bonded and plate forms a portion of a reservoir; providing at least one restoring force providing member positioned between the plates; placing two plates opposite each other; bonding the first and second films together to create a fluid-tight seal between the two plates; providing a mechanism for securing the plates to one another in a compressed mode wherein the at least one restoring force providing member is compressed; providing a mechanism for releasing the two plates in an extended mode wherein the at least one restoring force providing member is extended; and providing a conduit connecting the intake port that is connected to a catheter.

In still another aspect of the invention, a method for draining fluid from a patient includes: bonding a first film to a first plate and second film to a second plate so that each bonded film and plate forms a portion of a reservoir; providing at least one restoring force providing member positioned between the plates; placing two plates opposite each other; bonding the first and second films together to create a fluid and vacuum-tight seal between the two plates; providing a mechanism for securing the plates to one another in a compressed mode wherein the at least one restoring force providing member is compressed; providing a mechanism for releasing the two plates in an extended mode wherein the at least one restoring force providing member is extended; and connecting the intake port to a catheter in a patient so that a vacuum exerted on the intake port draws fluid from the patient.

Aspects of the present invention provide benefits and advantages that include an increased vacuum provided by restoring force providing members, increased compressibility and storage of the system, increased ease of use. Further, aspects of the present invention provide benefits in terms of the ease of manufacture of the system.

Additional advantages and novel features relating to the present invention will be set forth in part in the description that follows, and in part will become more apparent to those skilled in the art upon examination of the following or upon learning by practice certain aspects of the invention.

### BRIEF DESCRIPTION OF THE DRAWINGS

The present invention will become fully understood from the detailed description given herein below and the accompanying drawings, which are given by way of illustration and example only and thus not limited with respect to aspects of the present invention, wherein:

FIG. 1A is a schematic diagram of an exemplary system in an extended mode in which aspects of the present invention could be used in draining fluid from a patient;

FIG. 1B shows the exemplary system of FIG. 1A in a compressed mode;

FIG. 2A shows a closeup of a plate in another exemplary variation of the invention;

FIGS. 2B and 2C show an exemplary hook and loop configuration on a strap that may be used in conjunction with various aspects of the invention;

FIGS. 2D-2F show other exemplary systems that may be used in conjunction with various aspects of the invention;

FIGS. 3A and 3B show a close-up view of the system of FIG. 2A in the collapsed and expanded configurations, respectively;

FIGS. 3C and 3D show the results of optimization of parameters relating to various aspects of the invention;

FIGS. 4A and 4B show an exemplary apparatus that may be used to produce a system for fluid extraction in accordance with various aspects of the present invention;

FIGS. 5A and 5B show exemplary steps in a method to produce a system for fluid extraction according to aspects of the present invention;

FIGS. 6A and 6B show the resultant middle section that may be fabricated by the process shown in FIGS. 5A and 5B;

FIG. 7A highlights an exemplary cap and drain system that may be used with the present invention;

FIG. 7B shows a second exemplary cap and drain system with a second single-use cap;

FIG. 7C shows a third exemplary cap and drain system with a third single-use cap;

FIG. 8 shows another exemplary drain system based on piercing a portion of the system;

FIG. 9 shows yet another exemplary drain system also based on piercing a portion of the system;

FIG. 10 shows another exemplary drain system based on snapping the intake line at a weakened, perforated or brittle portion of the intake line;

FIGS. 11A and 11B show another exemplary drain system based on using a tab mechanism to open a hole in the system;

FIGS. 12A and 12B show another exemplary drain system based on using a tab mechanism to open a hole in the system;

FIG. 13 shows yet another exemplary drain system based on using a tab mechanism to open a hole in the system;

FIGS. 14A and 14B show another exemplary drain system based on a single-use nozzle to evacuate liquid from the system;

FIG. 15 shows yet another exemplary drain system based on using a tear strip to tear open a hole in the system;

FIGS. 16A and 16B show another exemplary drain system based on using a cover mechanism to open a hole in the system;

FIGS. 17A and 17B show another exemplary drain system based on using a tab mechanism to open a hole in the system;

FIG. 18 shows yet another exemplary drain system based on using a tear strip;

FIG. 19 shows yet another exemplary drain system based on using a valve;

FIG. 20 shows yet another exemplary drain system based on using a tear strip;

FIGS. 21A and 21B show another exemplary restoring force mechanism for use with the system;

FIGS. 22A-22C show a valve mechanism that may also be used in accordance with aspects of the present invention; and

FIGS. 23A-23D show another exemplary restoring force mechanism for use with the system;

FIG. 24 shows yet another exemplary drain system based on using a valve; and

FIG. 25 shows an exemplary Y-valve drainage system 2600 that may be used according to aspects of the present invention.

### DETAILED DESCRIPTION OF ASPECTS OF THE PRESENT INVENTION

Aspects of the present invention will now be described more fully hereinafter with reference to the accompanying drawings, in which variations and aspects of the present invention are shown. Aspects of the present invention may, however, be realized in many different forms and should not be construed as limited to the variations set forth herein; rather, the variations are provided so that this disclosure will be thorough and complete in the illustrative implementations, and will fully convey the scope thereof to those skilled in the art.

Unless otherwise defined, all technical and scientific terms used herein have the same meaning as commonly understood by one of ordinary skill in the art to which aspects of the present invention belong. The methods and examples provided herein are illustrative only and not intended to be limiting.

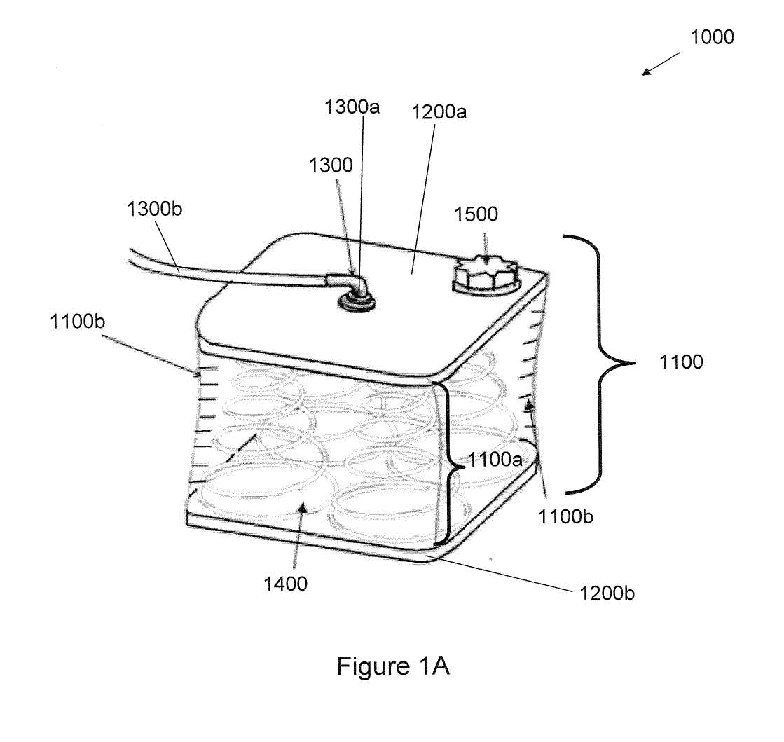

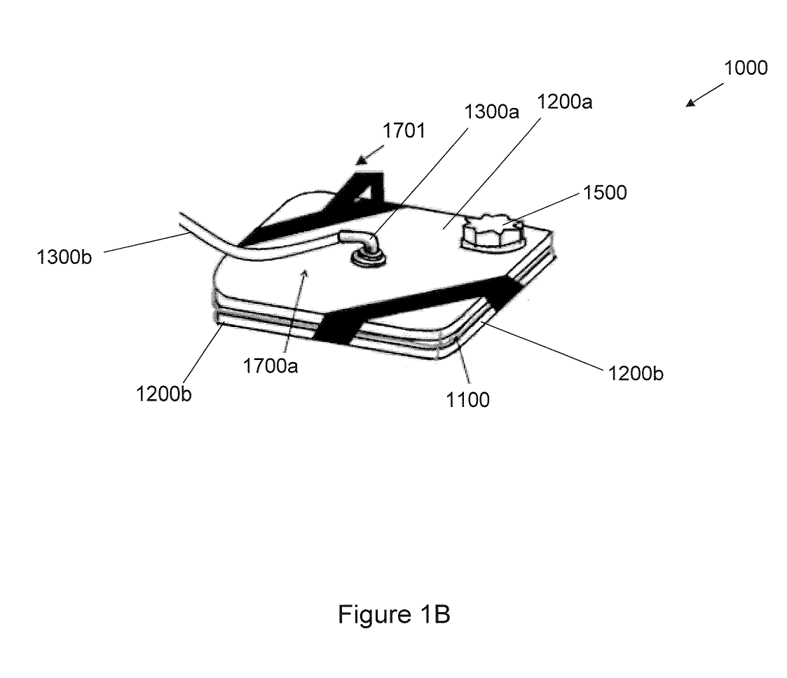

FIG. 1A is a schematic diagram of an exemplary system in an extended mode in which aspects of the present invention could be used in draining fluid from a patient. As shown in FIG. 1A, the system 1000 may include a main body 1100 that includes plates 1200a and 1200b. In between plates 1200a and 1200b, there may also be a middle section 1100a which may be sealed with the plates such that fluid contained within the middle section does not escape. Generally, the system 1000 is a container that serves to contain extracted fluid within the system 1000 and that is capable of sustaining negative pressure. Fluid from the patient may enter through the intake port 1300. Within the system 1000 there may be any suitable number of springs 1400 or other restoring force providing members that may be used to pull a vacuum to collect body fluid.

The system 1000 may be delivered in compressed form, as shown in FIG. 1B. In compressed form, the springs 1400 or other restoring force providing members are compressed in a state in which they store mechanical energy. Then, in order to draw a vacuum and initiate fluid suction, the patient need only to release the system from the compressed state shown in FIG. 1B so that the springs 1400 or other restoring force providing members press on or otherwise bias the plates 1200a and 1200b away from each other to expand the system 1000. As the system expands or relaxes to an extended state shown in FIG. 1A under the force of the springs 1400 or other restoring force providing members, the system can create a vacuum that draws the fluid. Once the liquid in the system 1000 has reached a user-defined maximal level, the system can then be emptied through the cap and drain 1500. Each of the components and variations of the components, as well as other aspects of the system 1000, will be explored below.

The springs 1400 or other restoring force providing members may take any suitable form such that they provide a force on or otherwise bias the plates 1200a and 1200b away from each other sufficient to pull a vacuum in the system 1000 as the springs 1400 or other restoring force providing members are allowed to expand. For example, it may be advantageous for the springs 1400 or other restoring force providing members to take the form of conical springs such that the springs take up a minimal amount of space in compressed form. Alternatively, the springs 1400 or other restoring force providing members may include other types of springs, including compression or leaf springs. Additionally, the springs 1400 or other restoring force providing members may include absorbent members such as sponges that collect fluid as they expand. The springs 1400 or other restoring force providing members may include still other restoring force providing mechanisms such as wound coils, levers or other suitable mechanisms. The springs 1400 or other restoring force providing members may be pre-set so that even when the system 1000 is in fully extended form (FIG. 1A) the springs 1400 or other restoring force providing members provide a force on plates 1200a and 1200b. That is, springs 1400 or other restoring force providing members may be pre-set so that even when the system 1000 is in a fully extended state (note that phrases “extended state,” “extended mode,” and “extended form” will be used interchangeably as will the phrases “compressed state,” “compressed mode,” and “compressed form”) the springs 1400 or other restoring force providing members are not fully extended. This baseline restoring force, i.e., the force provided by the springs 1400 or other restoring force providing members when the system 1000 is fully extended, may be useful for sustaining a sufficient vacuum when the system is in use to draw fluid. Further, the baseline restoring force may ensure that the system 1000 does not fail to provide a vacuum even as the system 1000 fills with fluid and that the provided vacuum remains relatively constant during fluid extraction.

Generally, the middle section 1100a shown in FIG. 1A is made of a flexible, transparent material, such as polymer or plastic. The material of the middle section 1100a is most commonly a film of such material, although the material of the middle section 1100a may include multiple layers, may be relatively thick and may also include sections that are thick. The middle section 1100a may also be made of material that is opaque or translucent. In many variations, the middle section 1100a is flexible such that it may expand or inflate in order to accommodate collected fluid. However, it may be advantageous for only a portion of the middle section 1100a to be flexible. Further, it may be advantageous in some variations for the middle section 1100a to contain portions that are rigid. In such variations, rigid portions of the middle section 1100a may be connected to one another via hinges or other flexible to allow the overall volume of the system 1000 to increase as fluid is collected. In any case, the edges or corners of the middle section 1100a may be transparent and marked with volume marks 1100b to indicate the volume of collected fluid, as shown in FIG. 1A.

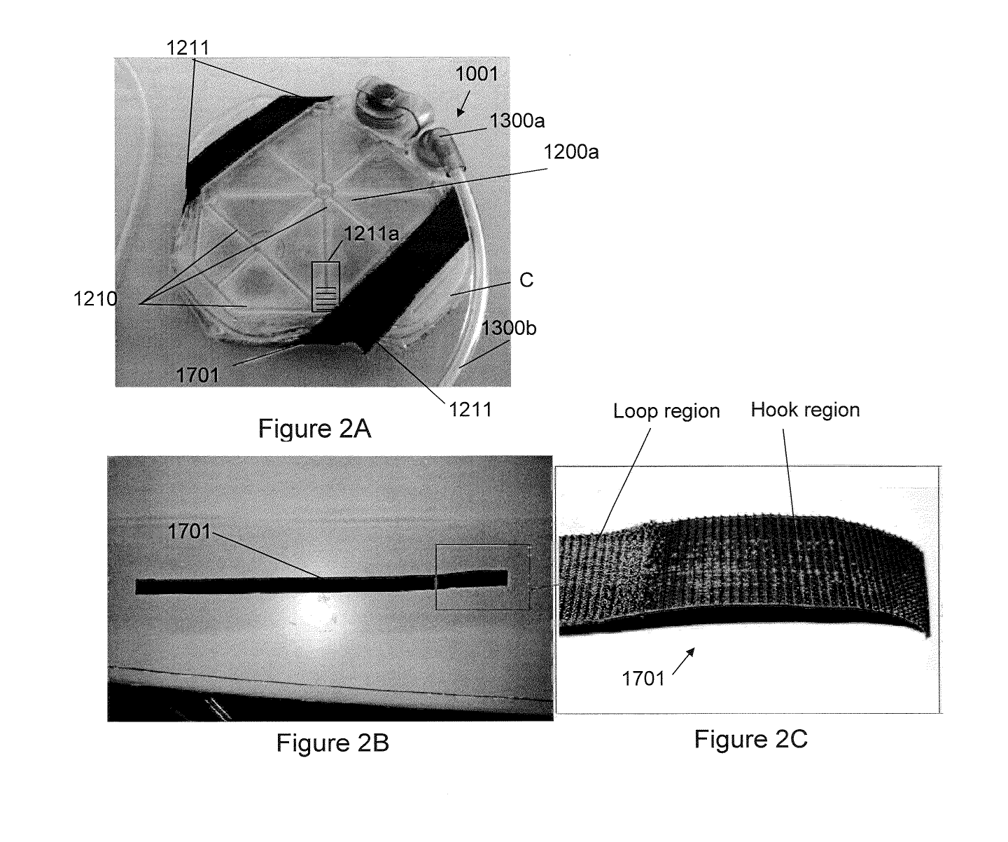

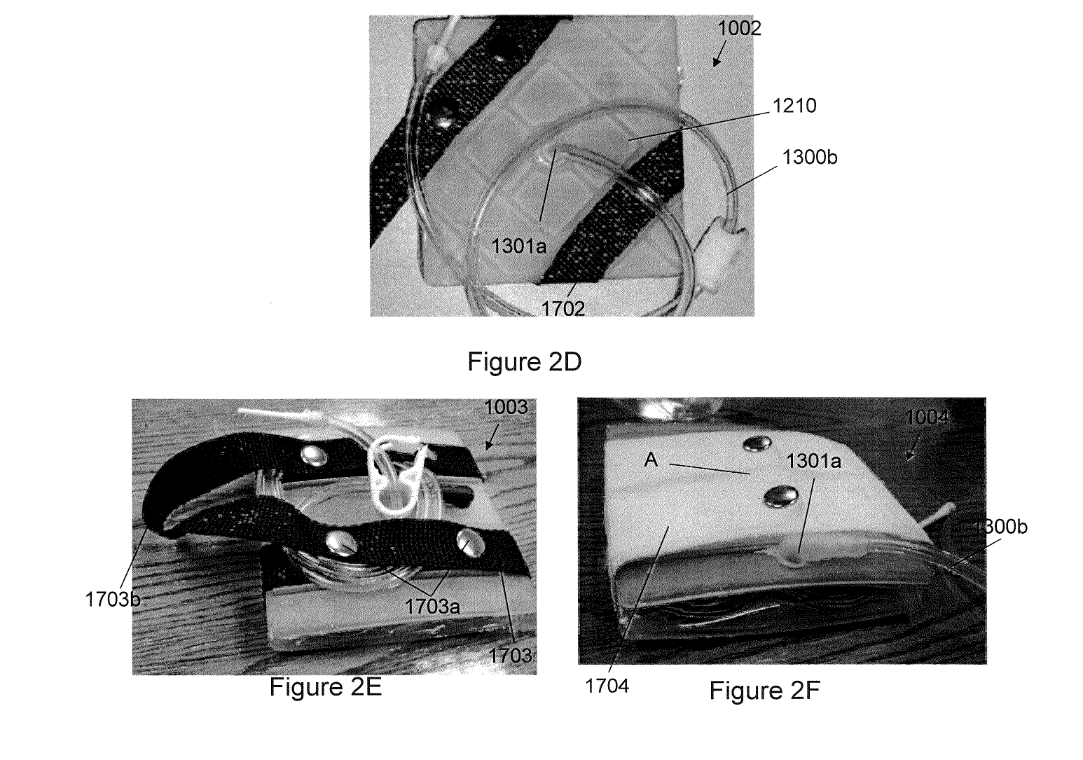

FIG. 2A shows a closeup of a plate 1200a in another exemplary variation of the invention. Generally, the plates 1200a and 1200b include rigid material, such as various plastics, glass, PMMA (Plexiglas), COC metal or other materials. The plates 1200a and 1200b may also include other materials such as paper, wire, rubber or other suitable non-rigid material. The plates 1200a may be entirely rigid, or they may include portions that are not rigid, including tearable seals, diaphrams, windows or other aspects. The plates 1200a and 1200b may be transparent or include transparent portions so that the level of liquid in the middle section 1100a can be ascertained visually from above or from other directions. As shown in FIG. 2A, the plates 1200a and 1200b may include rib structures 1210 for reinforcement or other purposes. For example, the rib structures 1210 may crisscross one of the surfaces of the plates 1200a and 1200b, as shown in FIG. 2A. The rib structures 1210 may crisscross one of the surfaces of the plates 1200a and 1200b, in any number of suitable patterns, including that shown in FIG. 2A. The rib structures 1210 may exhibit still other suitable patterns, including, for example, ringing the sides of the plates 1200a and 1200b. The rib structures 1210 may also, for example, provide structural support to the plates 1200a and 1200b to prevent them from bending, breaking or warping while experiencing the force provided by the springs 1400 or other restoring force providing members.

As shown in FIG. 2A, the plate 1200a may also in include a volume indicator 1211a. The volume indicator 1211a may, for example, be a pattern of markings on the side of the plate 1200a calibrated to give the volume of liquid contained in the exemplary system 1000. The volume indicator 1211a may include printed markings, raised markings or other suitable types of markings. In one variation, the volume indicator 1211a may be calibrated such that, upon completion of draining, a user could set the device on its side (e.g., so that it rests on the middle section 1100a) and read the volume of fluid through the plate 1200a using the volume indicator 1211a. In another variation, the volume indicator 1211a may be placed on a corner C of the plate 1200a such that volume may be measured similarly by resting the system 1000 on the corner C. It should be appreciated that the volume indicator 1211a may be placed in many other positions in any of the variations discussed herein on any of the plates or other aspects of the system 1000 or other systems and variations discussed herein.

It should be noted that, unlike many types of vacuum bottles that are sealed prior to sterilization, the system 1000 and other variations discussed herein may be sterilized by a variety of techniques. For example, the system 1000 and other variations described herein may be sterilized by the application of Ethylene Oxide gas, UV radiation, alcohol or other suitable sterilization procedure. Sterilization may then allow the collection of a sterile sample of the collected fluid for diagnostic purposes. The volume of the sterile sample can be, for example, measured using the volume indicator 1211a.

As shown in FIGS. 1A, 1B and 2A, one or more of the plates 1200a and 1200b may include an intake port 1300. The intake port 1300 may further include an elbow connector 1300a, as shown in FIG. 1A, 1B or 2A for connecting the intake port 1300 with a hose or intake line 1300b. The hose or intake line 1300b may then be connected to a patient via a catheter or other mechanism for inserting the line into or connecting to a patient. The intake port 1300 may also include any other type of suitable connector, such as, for example, the straight connector 1301a shown in FIG. 2D. The intake port 1300 may be located in any suitable position, including in the center of one of the plates 1200a, as shown in FIGS. 1A, 1B and 2D. However, the intake port 1300 may also be located elsewhere, such as on the side of one of the plates 1200a, as shown in FIGS. 2A and 2F. In addition or in alternative to the above configurations, it may also be advantageous for the intake port 1300 to be placed on other portions of the system 1000 such as on middle section 1100a.

Although not shown, the intake port 1300 may further include a valve for various purposes, including for preventing back flow of collected liquid or for maintaining negative pressure in the system 1000. For example, the intake port 1300 may include a one-way valve such as a check valve or a diaphragm valve. Alternatively, the intake port 1300 may include a valve that allows flow in more than one direction, such as, for example, a gate valve, plug valve or globe valve. The intake port 1300 may also include one elbow connector 1300a or a plurality of elbow connectors 1300a as well as other suitable types of connectors. Although only one hose or intake line 1300b is shown in FIGS. 1A and 1B, it is to be understood that multiple hoses 1300b may connect to the system. For example, it is possible to connect a plurality of hoses 1300b in order to draw fluid from multiple patients or from multiple sites on the same patient, simultaneously. A plurality of such multiple hoses 1300b may, for example, be connected through the same intake port 1300 or through additional intake ports 1300.

FIGS. 1B and 2A also show straps that may be included with the systems. The exemplary straps 1701 generally hold the springs 1400 or other restoring force providing members in a compressed mode (FIG. 1B) for storage or transport. The exemplary straps 1701, for example, shown in FIGS. 1B and 2A include a hook and loop configuration reversibly binding the straps to one another, or parts of one of the straps to other parts of the same strap. A strap with a hook and loop configuration for binding one part of itself to another is shown in more detail in FIGS. 2B and 2C. Hook and loop configurations that may be used with the instant invention include commercially available Velcro and other similar configurations that involve creating to adjacent contact portions on the strips to be bound and including loops on one of the contact portions and hooks on the other. In order to bind the two contact portions together, they can be pressed together such that some of the loops are ensnared in some of the loops. Other configurations are also possible that include patterned hook and loop configurations and/or clasps, fasteners or clips. Generally, the hook and loop configuration is such that it is stronger in shear than in perpendicular tension. In this case, the user can pull apart two of the straps with relative ease, yet they remain adhered to one another unless they experience force in the tensile direction. In this way, the straps 1701 can be suitably strong to hold the system 1000 in compressed mode (FIG. 1B), yet the straps 1701 allow the user to release of the system from compressed mode relatively easily (e.g., by simply pulling apart the hook and loop portions of the straps 1701). This can be particularly advantageous when patients in a weakened condition need to activate the system 1000.

FIG. 2A also shows that the edges of the plates 1200a and 1200b may be scalloped 1211 in order to accommodate the straps 1701. In general, the scalloping 1211 may be gradual, as shown in FIG. 2A, or it may have a more severe shape. For example, the scalloping 1211 may take the shape of a groove that fits the straps 1701 precisely so as to prevent lateral movement of the straps 1701. The scalloping 1211 may further include a loop, ring or clip for fixing the straps 1701 into place. The plates 1200a and 1200b may have any suitable shape for the application. Suitable shapes include: octagonal, rectilinear, or rounded square, disk shapes or other rounded shapes.

FIGS. 2D-2F show other exemplary systems that may be used in conjunction with various aspects of the invention. As shown in FIGS. 2D-2F, the exemplary straps 1702-1704, for example, shown in FIGS. 2D-2F include a snap configuration reversibly binding the straps to one another. Snap configurations that may be used with the instant invention include commercially available snaps, buttons and other similar fasteners that involve mating two or more portions of the fasteners such that the two or more portions bind to one another. Other configurations are also possible including pins, zippers, clips, tabs or and loop configurations and/or clasps, fasteners or clips. Generally, the snap configuration is such that it is stronger in sheer than in tension. In this case, the user can pull apart the mating portions with relative ease, yet they remain adhered to one another unless they experience a relatively large force in the direction that pulls them apart. Additionally, the snaps may be activated or released by a turning release mechanism, releasing springs, buckles or other suitable release mechanisms. In general, the straps may include any of the configurations shown, as well as any other suitable configuration that is able to counteract the biasing force of the springs 1400 or other restoring force providing members that hold the system in the compressed mode in FIG. 1B.

FIGS. 2E and 2F show two other variations of the system 1003 and 1004, respectively. FIG. 2E shows a variation of exemplary straps 1703 that include snaps 1703a as well as a handle 1703b. The handle 1703b may allow the user increased leverage for separating the two mating portions of the snaps 1703a in order to release the exemplary straps 1703. This increased leverage may be particularly important for patients in a weakened state. FIG. 2F shows another variation in which exemplary straps 1704 include an increased contact area A between the straps 1704. The increased contact area A may, for example, include snaps and/or another mechanism, such as the hook and loop mechanism discussed above. The increased contact area A may enhance bonding between the straps and, therefore, more securely fix the system 1000 in compressed mode.

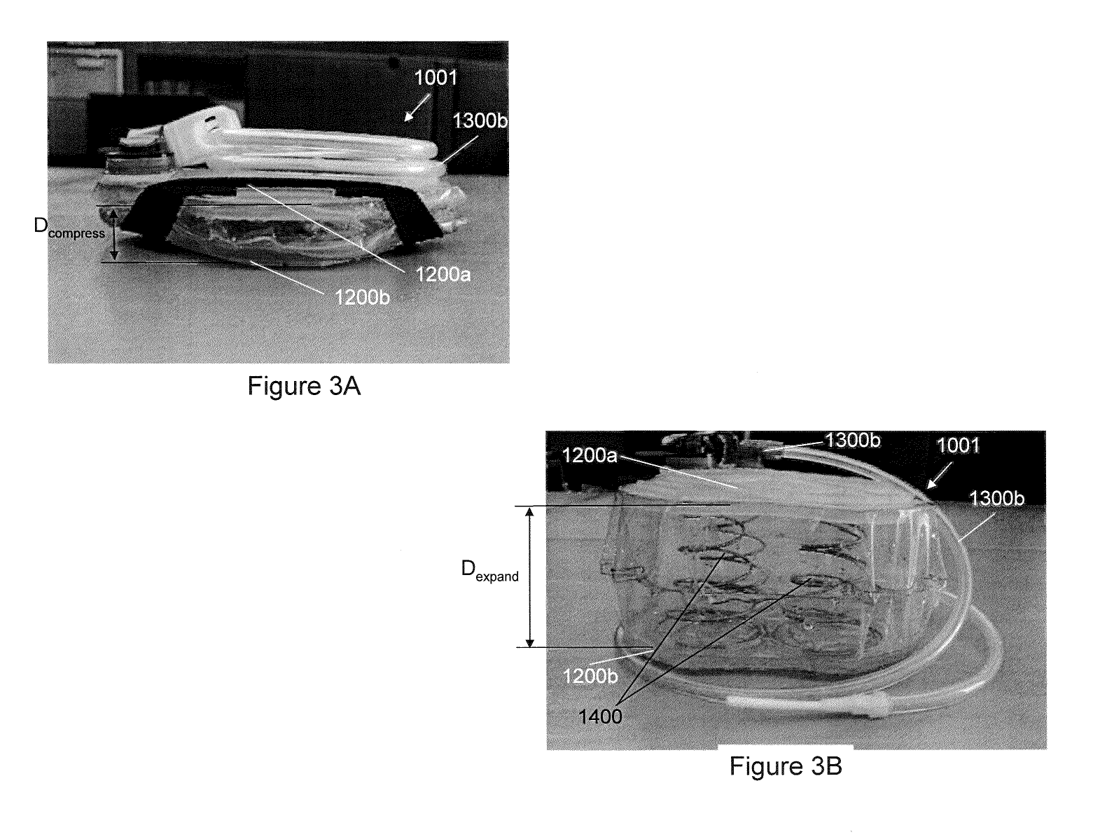

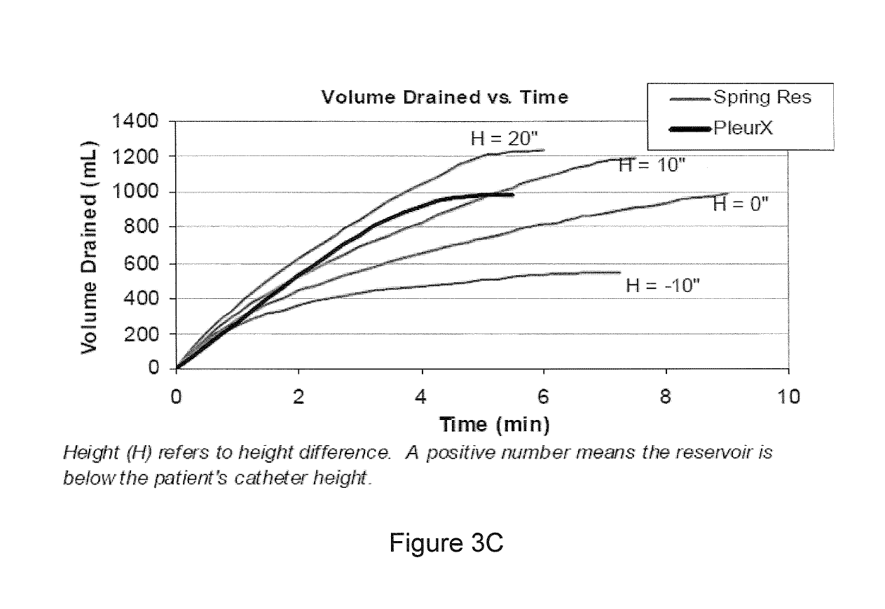

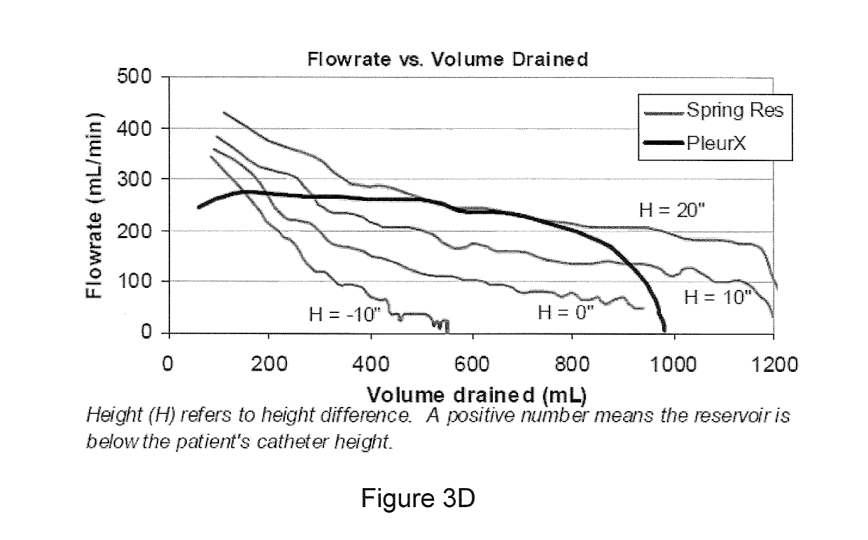

FIGS. 3A and 3B show a close-up view of the system 1001 of FIG. 2A in the collapsed and expanded configurations, respectively. As shown in FIG. 3A, when the system is fully compressed it assumes the height D_{compress} between the two plates 1200a and 1200b. As shown in FIG. 3B, when the system is fully expanded it assumes the height D_{expand} between the two plates 1200a and 1200b. In general, it is advantageous to minimize the compressed mode ratio, defined as D_{compress}/D_{expand}. Systems with a lower compressed mode ratio D_{compress}/D_{expand} are generally more easily stored, stacked and delivered in bulk to a patient. A decreased compressed mode ratio D_{compress}/D_{expand} generally implies a greater vacuum capability of the system 1001 because the compressed mode ratio D_{compress}/D_{expand} is inversely related to the maximum force delivered by the springs 1400 or restoring force providing members, all other things (e.g., the spring constants, etc.) being equal. Decreasing the compressed mode ratio D_{compress}/D_{expand} may also make it possible to deliver an increased number of systems 1001 in the same package for the convenience of the user, as well to save time and shipping cost. Further, a lower the compressed mode ratio D_{compress}/D_{expand} can mean an increase in the number of systems 1001 that can be stored in a patient's environment. The latter may be an environment with extremely limited space such as a home, hospital bedroom, or recovery room.

FIGS. 3A and 3B show an exemplary system 1001 in which the compressed mode ratio D_{compress}/D_{expand} is around 0.25. In principle, however, even lower compressed mode ratios D_{compress}/D_{expand} are possible. Generally, the compressed mode ratio D_{compress}/D_{expand} is influenced by a combination of factors including the compressibility of the springs 1400 or restoring force providing members. In particular, conical springs are advantageously used in the present invention because such springs tend to be particularly compressible and can lead to decreased compressed mode ratios D_{compress}/D_{expand}. However, other types of springs 1400 or restoring force providing members also may be used with the present invention.

FIGS. 3C and 3D show the results of optimization of parameters relating to various aspects of the invention. Parameters of the present invention that my be optimized for maximum flow rate include: the type of springs 1400 or other restoring force providing members (including such variables as the type of wire used in the springs, etc.), the height H, diameter D and other parameters to prevent the buckling or permanent deformation of the springs 1400 or other restoring force providing members under loading. It is to be understood that the optimization of all such parameters for uses discussed herein and other suitable uses is within the context of the present invention.

Buckling or permanent deformation could reduce the flow rate of liquid into the system 1000. Negative pressure or vacuum generated by the force of the springs 1400 or other restoring force providing members against plates 1200a and 1200b, ultimately providing the flow of liquid into the system 1000, can depend on the force provided by each of the springs 1400 or other restoring force providing members and the area A1 over which that force is applied (e.g., on the top plates 1200a and 1200b). A smaller area A1, all other things being equal, results in a greater pressure (P=F/A). Decreasing the area A1, all other things being equal, would result in a decreased pressure. In this situation, if it were desired to draw the same fluid volume, the distance between the plates 1200a and 1200b could be increased. However, increasing the distance between the plates 1200a and 1200b can decrease the overall stability of the system 1000 by, among other things, increasing the tendency for buckling or permanent deformation of the springs 1400 or other restoring force providing members. Increasing the distance between the plates 1200a and 1200b may also increase spring buckling, variations in the flow rate profile, or create difficulties in manufacturability. The size and shape of the system 1000, among other things, can be altered to optimize these factors. The flow rate, in particular, may be dependent on suction pressure provided by the system 1000, among other things, as well as the fluid path from the patient to the system. The type of tubing used and its length can be chosen, for example, to yield an optimal flow rate. For example, the type of tubing used and the length of the tubing can be chosen such that, for example, the length is as long as possible without causing substantial kinking of the tubing.

FIG. 3C shows the variation in flow rate of the system 1000 with H, where H is the height difference between the system 1000 and the catheter. Drainage time can be particularly important to active patients who do not wish to spend an excessive amount of time for fluid draining. The flow rate of the system 1000 is compared to the flow rate of the Pleurex evacuators (U.S. Pat. No. 5,484,401) that include a pre-loaded vacuum bottle and do not include springs or other restoring force providing members. As FIG. 3C shows, 1000 ml of pleural or peritoneal fluid can be drained in 15 minutes or less while the system 1000 is at equal height with the catheter (H=0). The flow rate of the system 1000 of the present invention can be faster than the flow rate of other commercially available systems. Drainage time can be a function of flow rate. Flow rate, in turn, can be a function of the negative pressure generated by the system 1000 as well as the fluid pathway between the system 1000 and the catheter. Particularly high negative pressures (i.e., a strong vacuum) can be difficult to achieve because of the dependence of such pressures on a balance between the spring force or restoring force and surface area over which that surface force is applied. Stronger springs often require more robust or rigid components (such as the top and bottom plates 1200a 1200b). FIG. 3D shows that the drainage time can also be slowed or sped up by using gravity by altering the distance H between the system.

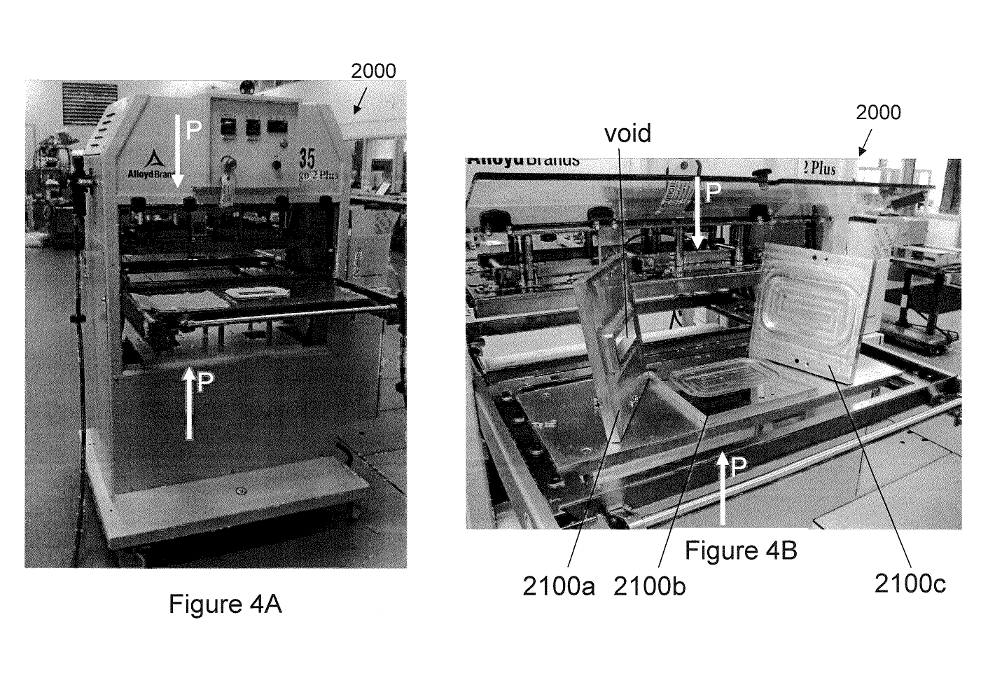

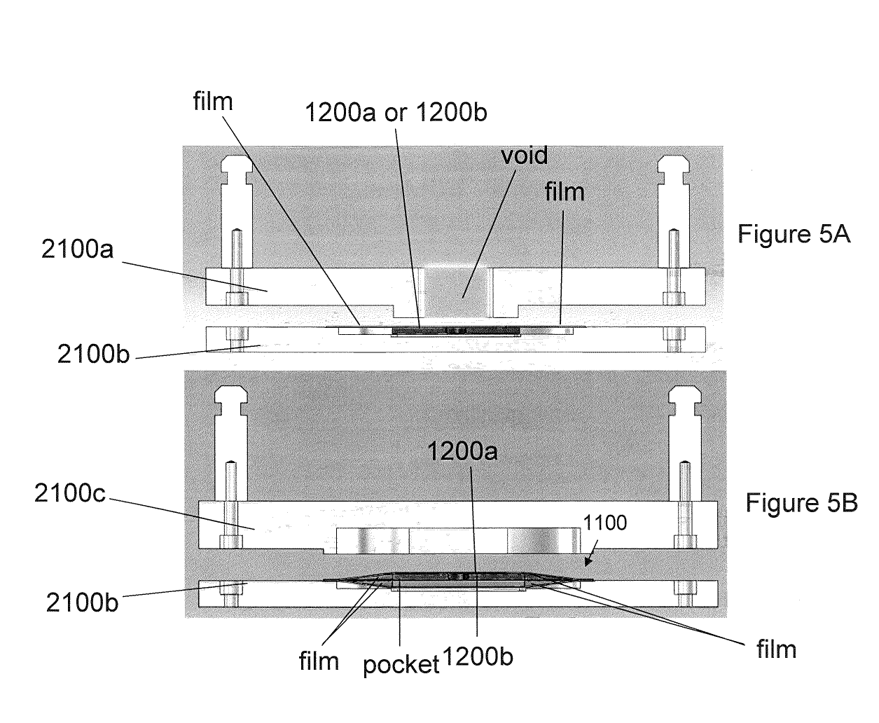

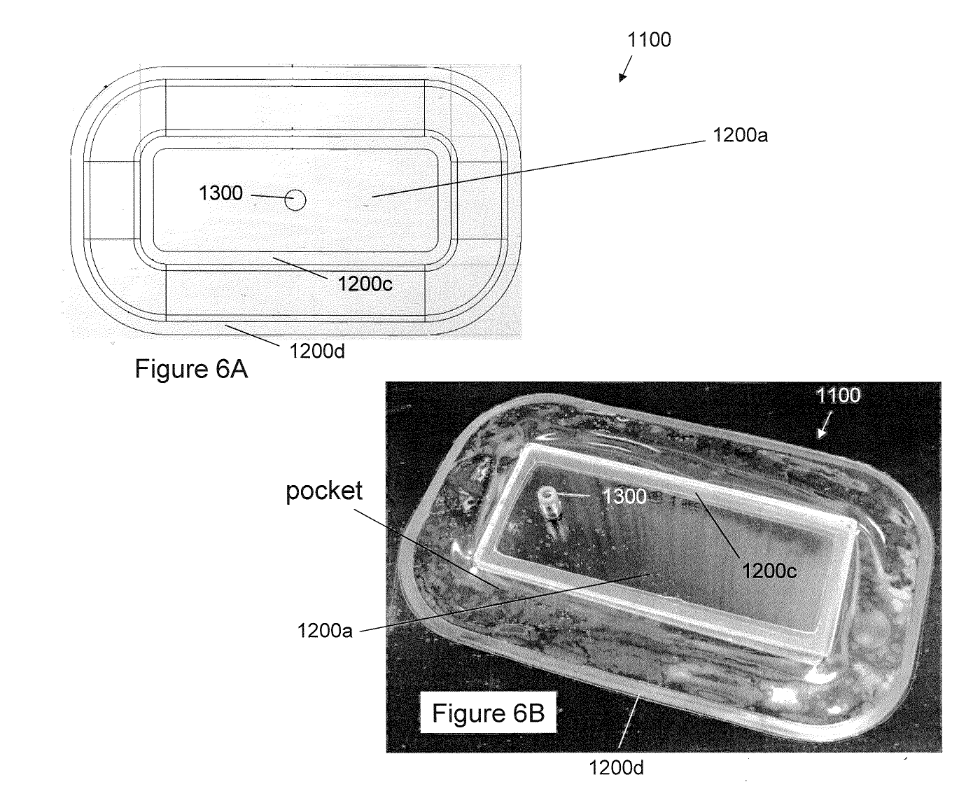

FIGS. 4A and 4B show an exemplary apparatus that may be used to produce a system for fluid extraction in accordance with various aspects of the present invention. FIGS. 5A and 5B show exemplary steps in a method to produce a system for fluid extraction according to aspects of the present invention. FIGS. 6A and 6B show the resultant middle section 1100a that may be fabricated by the process shown in FIGS. 5A and 5B.

As shown in FIG. 4A, a heat press 2000 can be used to seal the main body 1100 of the system 1000. The heat press 2000 may apply heat and pressure in a number of directions, including the direction P shown in FIGS. 4A and 4B. Generally, the heat press 2000 will have a number of implements 2000a-2000c, as shown in FIG. 4B. Although three implements 2000a-2000c are shown in FIG. 4B, it is to be understood that this is merely exemplary. In principle, any suitable number of implements can be used to produce the system 1000 using any suitable number of steps. Further, the implements are shown in FIG. 4B placed on the top of the heat press 2000 for viewing. In operation, the implements 2101a-2101b are generally placed such that the pattern surfaces face the direction of applied pressure P. However, in principle, the implements 2101a-2101b may be oriented in any suitable direction during fabrication of the system 1000.

As shown in FIG. 5A, an exemplary first step in the fabrication of the system 1000 may be to bring an implement 2100a in contact with implement 2100b in a manner that seals a plate 1200a or 1200b to a film. In this or other ways, each half of the middle section 1100a may be fabricated independently. The film may be placed adjacent to the plate 1200a or 1200b, as shown in FIG. 5A. The film then serves as the flexible exterior of the middle section 1100a shown in FIG. 1A. The film may comprise any of the materials discussed herein relating to the middle section 1100. Generally, the plate 1200a or 1200b is placed in contact with the film and the heat press 2000 is used to apply heat and pressure in direction P in order to bring implement 2100a in contact with the film, as shown in FIG. 5A. The heat and pressure applied by the heat press 2000 may be sufficient to fuse, melt or weld the plate 1200a or 1200b with the film. In this manner one side of the middle portion 1100 (FIG. 1A) may be fabricated. The heat and pressure applied by the heat press 2000 is generally applied along the periphery 1200c of the plate 1200a (FIG. 6A) where the film overlaps the plate 1200c. However, the heat and pressure may be applied in any suitable direction.

The plate 1200a and the film may be fused directly, or there may be a layer of adhesive placed between the film and the plate 1200a prior to the application of heat and pressure by the heat press 2000. In order for the film to adhere to the plate 1200a, the materials must be compatible. Any suitable material for the film, plate or adhesive may be used including various plastics, thermoplastics, epoxies or other suitable materials.

Once both halves of the middle section 1100a are fabricated in the manner shown in FIG. 1A, or in a like manner, they may be fused together by the heat press 2000 to form middle section 1100a in the manner shown in FIG. 5B. As shown in FIG. 5B, each of the halves of the middle section 1100a are placed on top of one another on implement 2100b. Note that FIG. 5B shows a pocket between the two halves of the mid-section, indicating that each half is not adhered to the other prior to the application of the heat press. Although not shown in FIGS. 5A, 6A and 6B, the pocket may contain a variety of components, including the springs 1400 or other restoring force providing members. The heat press 2000 brings implements 2100b and 2100c together and applies heat and pressure in the direction P (FIGS. 4A and 4B) that may be sufficient to fuse, melt or weld the two sides of the film attached to plates 1200a and 1200b, respectively, to one another. The heat and pressure applied by the heat press 2000 is generally applied along the periphery 1200d of the film (FIGS. 6A and 6B) where the films from the two halves overlap. In order for the film to adhere to one another, the films must be compatible. The films may be fused directly, or there may be a layer of adhesive placed between the films prior to the application of heat and pressure by the heat press 2000. Any suitable material for the film or adhesive may be used including various plastics, thermoplastics, epoxies or other suitable materials.

It is to be understood that, while a heat press is discussed above, the system 1000 can be fabricated using a number of different sealing methods. The sealing methods include, but are not limited to: adhesive bonding, laser welding, ultrasonic welding, etc.

As shown in FIGS. 4A-6B, the main body 1100 may be pre-fabricated prior to the addition of other components such as the hose or intake line 1300b, the straps 1701, etc. Although not shown in FIGS. 4A-4D, generally the springs 1400 or other restoring force providing members will be included in the main body 1100 during fabrication. That is, the springs 1400 or other restoring force providing members are typically mounted to the plates 1200a and 1200b prior to the manufacturing step shown in FIG. 5B.

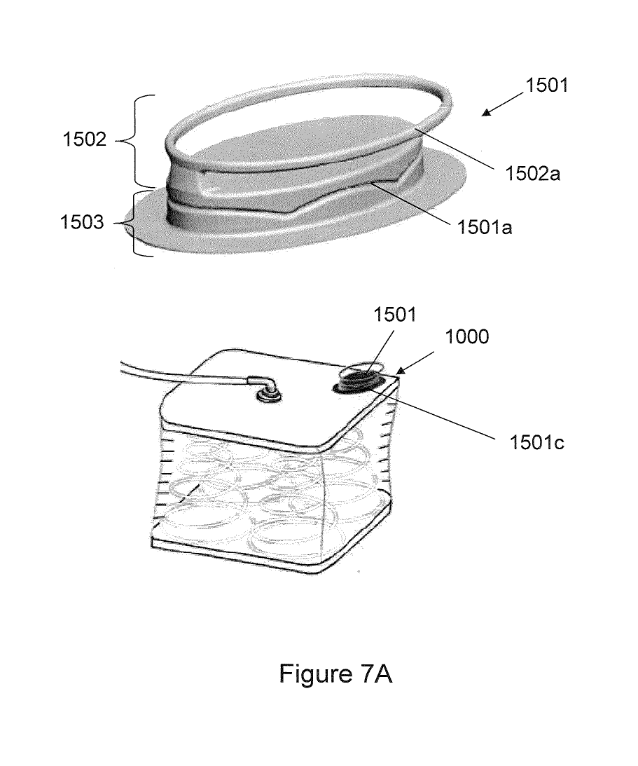

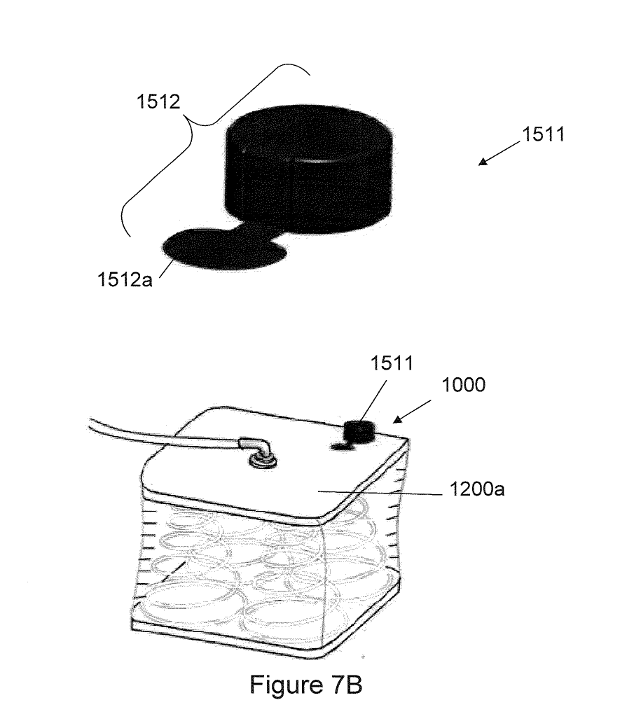

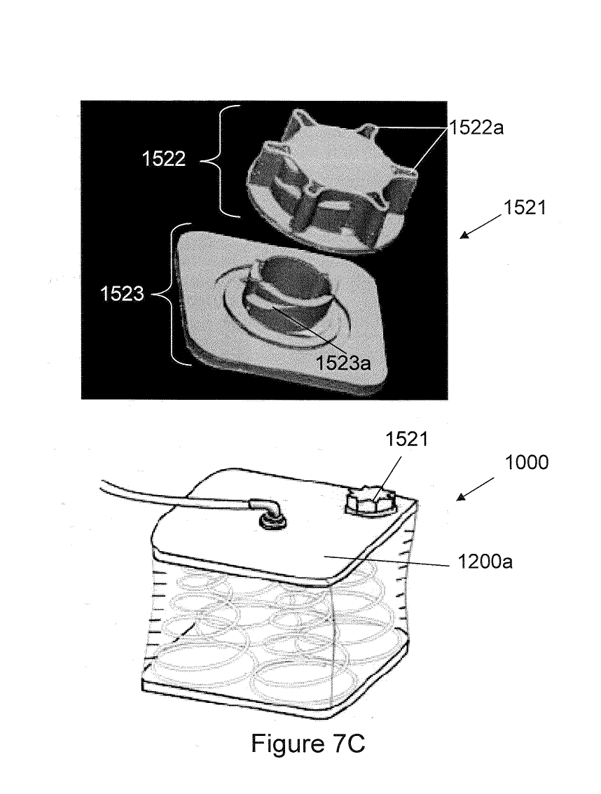

FIG. 7A highlights an exemplary cap and drain system 1501 that may be used with the present invention. Although in principle it would be possible to drain and re-use the system 1000 or other systems discussed herein after each use, it can be advantageous to discourage re-use by the user. This is because handling of body fluids by a user is generally dangerous and unhealthy, particularly if the user is a not a medical professional. Further, if the system is re-used but not properly cleaned between each use, a contamination risk can arise and can be particularly dangerous to patients already suffering from the kinds of ailments treated with system 1000. One way to obviate these dangers, difficulties and risks is to ensure that the system 1000, and other systems discussed herein, are disposable and will be discarded after use. In order to ensure this, a single-use cap and drain system, such as that shown in FIG. 7A, may be used.

FIG. 7A shows an exemplary cap and drain system 1501 with a single-use cap. As shown in FIG. 7A, the exemplary cap and drain system 1501 may be divided into two sections, a ring section 1502 and a fixed section 1503. Generally, the fixed section 1503 is permanently fixed to the system 1000, although it may be advantageous in some variations for the fixed section 1503 to be removable. The ring section 1502 includes a ring 1502a that may be grasped and pulled relatively easily by the user. When the user grasps and pulls the ring 1502a, the user may tear the exemplary cap and drain system 1501 along the seam 1501a. The seam 1501a can, for example, be perforated or deliberately weakened so that it can be easily torn. One method of deliberately weakening the seam 1501a, for example, includes thinning the material of the exemplary cap and drain system 1501 around the seam 1501a. Another includes chemically weakening the area around the seam 1501a. Any suitable method for creating a user-tearable seal may be implemented in conjunction with the present invention. The exemplary cap and drain system 1501 may be fabricated from any suitable material, including plastics, metals or metal foil. The ring 1501a may have any suitable shape such that the user can pull it. It may, for example, have the hoop shape shown in FIG. 7A. The ring 1501a may also include multiple finger holes, or it may include a gripping bar molded to fit in between a user's fingers when the user clenches his/her fingers around the gripping bar.

As shown in FIG. 7A, the exemplary cap and drain system 1501 may further include a membrane or weak valve 1501c, for example, underneath the ring section 1502 or at another suitable location. The membrane or weak valve 1501c may prevent reflux from occurring when the ring section 1502 is removed. The cracking pressure of the membrane or valve 1501c is generally high enough that fluid in the system 1000 does not easily spray out of the system 1000 once the ring section 1502 has been removed. On the other hand, the cracking pressure of the membrane or valve 1501c is generally low enough so that turning the system 1000 upside down and/or and squeezing the walls of the system 1000 to create positive pressure may add enough pressure to allow fluid to pass through the membrane or weak valve 1501c. It should be understood that the membrane or weak valve 1501c may be added to any of the variations of the invention discussed herein.

FIG. 7B shows a second exemplary cap and drain system 1511 with a second single-use cap 1512. The exemplary cap and drain system 1511 may be divided into two sections, a tab section 1512 and a fixed section (not shown). Generally, the fixed section is permanently fixed to the system 1000, although it may be advantageous in some variations for the fixed section to be removable. Further, the fixed section may be a portion of the plate 1200a. Although not shown, the tab section 1512 is usually attached to the fixed section in such a way as to form a liquid-tight seal. The attachment between the fixed section and the tab section 1512 may include a plastic or rubber seal as well as any other suitable type of seal. The tab section 1512 includes a tab 1512a that may be grasped and pulled relatively easily by the user. When the user grasps and pulls the tab 1512a the user may tear or break attachment between the fixed section and the tab section 1512. The second exemplary cap and drain system 1511 may be fabricated from any suitable material, including plastics, metals or metal foil. The tab section 1512 and the tab 1512a may have any suitable shape such that the user can pull it. It may, for example, have the hoop shape shown in FIG. 7B or it may have a ring shape shown in FIG. 7A. The tab section 1512 and the tab 1512a may also include multiple finger holes, or it may include a gripping bar molded to fit in between a user's fingers when the user clenches his/her fingers around the gripping bar.

FIG. 7C shows a third exemplary cap and drain system 1521 with a third single-use cap 1522. The third exemplary cap and drain system 1521 may be divided into two sections, a cap section 1522 and a fixed section 1523. Generally, the fixed section 1523 is permanently fixed to the system 1000, although it may be advantageous in some variations for the fixed section 1523 to be removable. Further, the fixed section 1523 may, in fact, be a portion of the plate 1200a. Although not shown, the cap section 1522 is usually attached to the fixed section 1523 in such a way as to form a liquid-tight seal. The liquid-type seal may be accomplished, for example, via screwing the cap section 1522 onto the fixed section 1523 using threads 1523a. The threads 1523a can be such that the cap section 1522 may be screwed onto the fixed section 1523, but may not be screwed off or removed without breaking the threads 1523a or rendering the threads 1523 inoperable. The attachment between the fixed section 1523 and the cap section 1522 may include a plastic or rubber seal as well as any other suitable type of seal. The cap section 1522 may include grips 1522a that may be grasped and used to screw the cap section 1522 relatively easily by the user. When the user grasps and pulls the grips 1522a the user may tear or break an attachment between the fixed section 1523 and the cap section 1522. The third exemplary cap and drain system 1521 may be fabricated from any suitable material, including plastics, metals or metal foil. The cap section 1522 may have any suitable shape such that the user can grasp and rotate it. It may, for example, have the star shape shown in FIG. 7C, a ring or other shape. The cap section 1522 may also include multiple finger holes, or it may include a gripping bar molded to fit in between a user's fingers when the user clenches his/her fingers around the gripping bar.

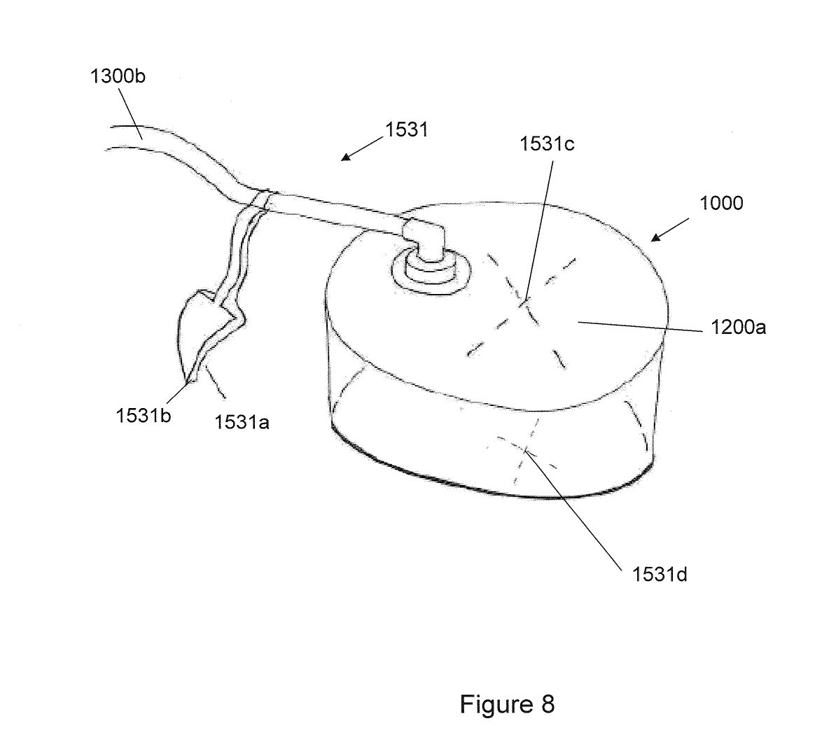

FIG. 8 shows another exemplary drain system 1531 based on piercing a portion of the system 1000. As shown in FIG. 8, the exemplary drain system 1531 includes a piercing tool 1531a that may be attached to the hose or intake line 1300b. Alternatively, the piercing tool 1531a may be completely unattached to the system 1000, or may be attached to any other suitable portion of the system 1000. Generally, the user may take a sharp edge 1531b of the piercing tool 1531a and use it to pierce a portion of the system at a particular location 1531c. Although, FIG. 8 shows the piercing location 1531c on the plate 1200a, the piercing location 1531c may be any suitable portion of the system 1000. For example, piercing may occur at a side location 1531d of the system. Once the system 1000 has been pierced by the user, the piercing tool 1531a may be removed and the system 1000 may subsequently emptied of fluid through the hole left in the piercing location. Since the piercing creates a permanent hole in some portion of the system 1000, it automatically renders the system unusable. Therefore, the exemplary drain system 1531 is a single-use system that may include the associated advantages discussed above.

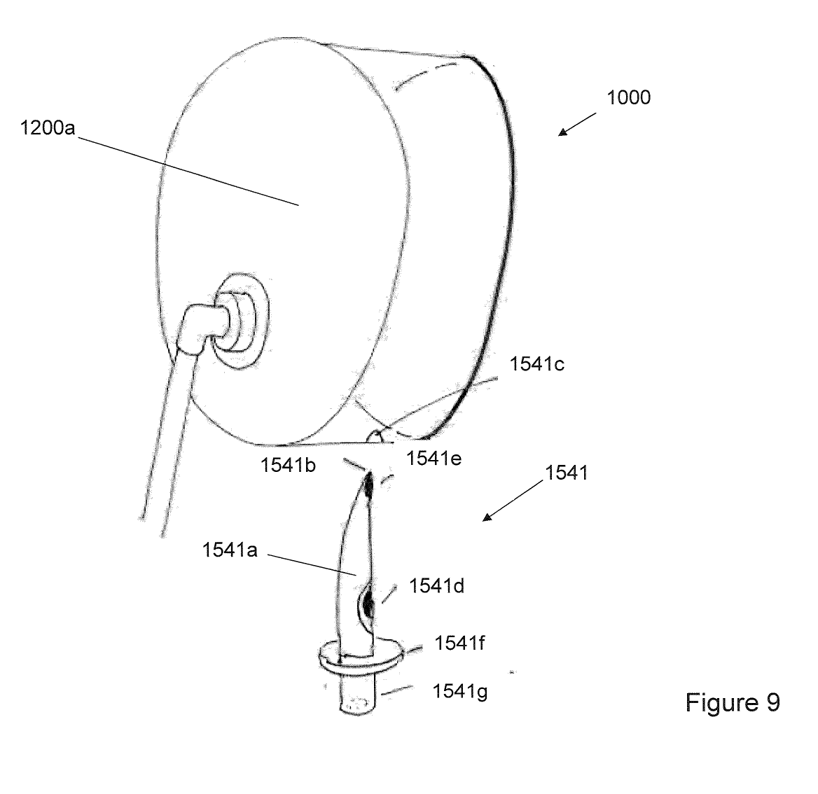

FIG. 9 shows another exemplary drain system 1541 also based on piercing a portion of the system 1000. As shown in FIG. 9, the exemplary drain system 1541 includes a piercing tool 1541a that may include a drain port 1541d connected to an intake hole 1541e. Generally, the user may take a sharp edge 1541b of the piercing tool 1541a and use it to pierce a portion of the system 1000 at a particular location 1541c, for example. The piercing tool 1541a may also include a stopper 1541f that controls the amount of the piercing tool 1541a inserted into the system 1000 and, therefore, the puncture or hole created by the tool. The user may manipulate the piercing tool 1541a by grasping the handle 1541g. Although FIG. 9 shows a piercing location 1541c on a side of the system 1000, the piercing location 1541c may be any suitable portion of the system 1000. Once the system 1000 has been pierced by the user, the piercing tool 1541a may remain in system 1000 such that the intake hole 1541e is in communication with fluid in the interior of the system 1000 while the drain port 1541d remains outside of the system 1000. In this configuration, fluid in the system 1000 may subsequently drain from the intake hole 1541e through the drain port 1541d. Since the piercing creates a permanent hole in some portion of the system 1000, the system 1000 cannot be re-used. Therefore, the exemplary drain system 1541 is a single-use system that may include the associated advantages discussed above.

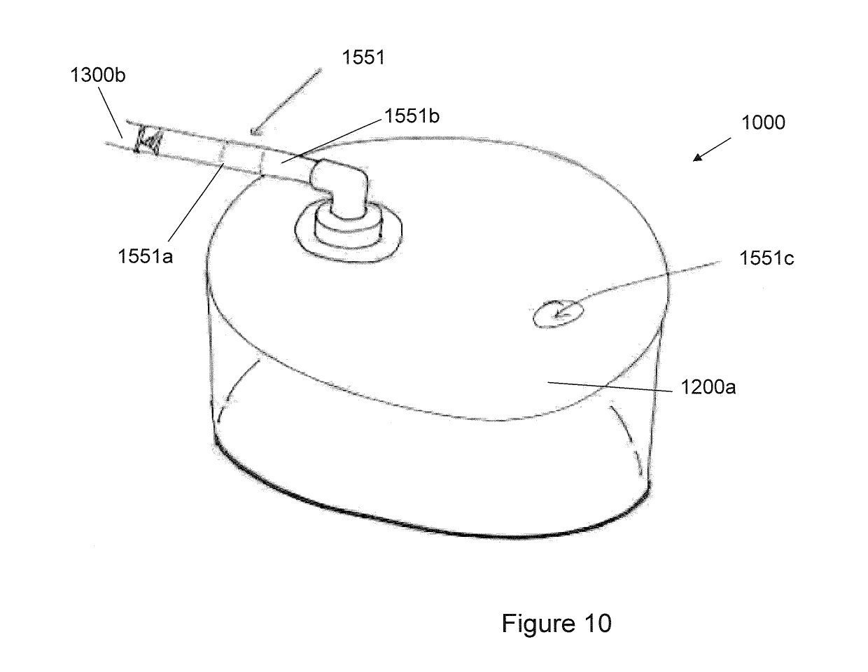

FIG. 10 shows another exemplary drain system 1551 based on snapping the intake line 1300b at a weakened, perforated or brittle portion of the intake line 1551a. The user may snap the intake line 1300b by, amongst other things, bending the intake line 1300b at the weakened, perforated or brittle portion of the intake line 1551a. Subsequently, the user can then use the remaining portion of the intake line 1551b as a spout. The user may, for example, pour out the liquid contents of the system 1000 through the remaining portion of the intake line 1551b and down a drain or in a waster receptacle. FIG. 10 also shows an optional one-way valve 1551c that can be used to compress the system once it has been evacuated of liquid or fluid. The one-way valve 1551c can, for example, allow the expulsion of air in the system 1000 after it has been evacuated of fluid so that the system 1000 can be crushed to a state resembling the compressed form, as shown in FIG. 1B. Since snapping the intake line 1300b creates a permanent hole in the system 1000, the system 1000 cannot be re-used. Therefore, the exemplary drain system 1551 is a single-use system that may include the associated advantages discussed above.

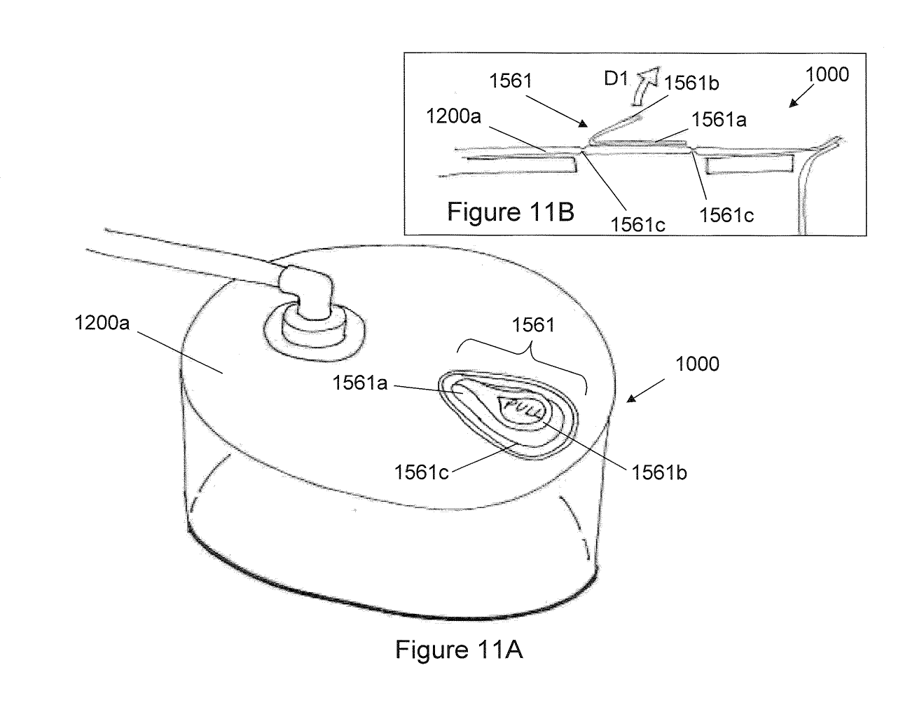

FIGS. 11A and 11B show another exemplary drain system 1561 based on using a tab mechanism 1561a to open a hole in the system 1000. After the user lifts the tab 1561b and pulls it along direction D1 shown in FIG. 11B, amongst other things, a hole opens up in system around seam 1561c. Generally, the seam 1561c will form a ring, or other shape, around the tab, as shown in FIG. 11A. However, any suitable seam 1561c configuration can be used in the context of the present invention. Subsequent to pulling the tab 1561b and opening a hole in the system 1000, the user may pour out the liquid contents of the system 1000 down a drain or in a waster receptacle. Since the user creates a permanent hole in the system 1000, the system 1000 cannot be re-used. Therefore, the exemplary drain system 1561 is a single-use system that may include the associated advantages discussed above.

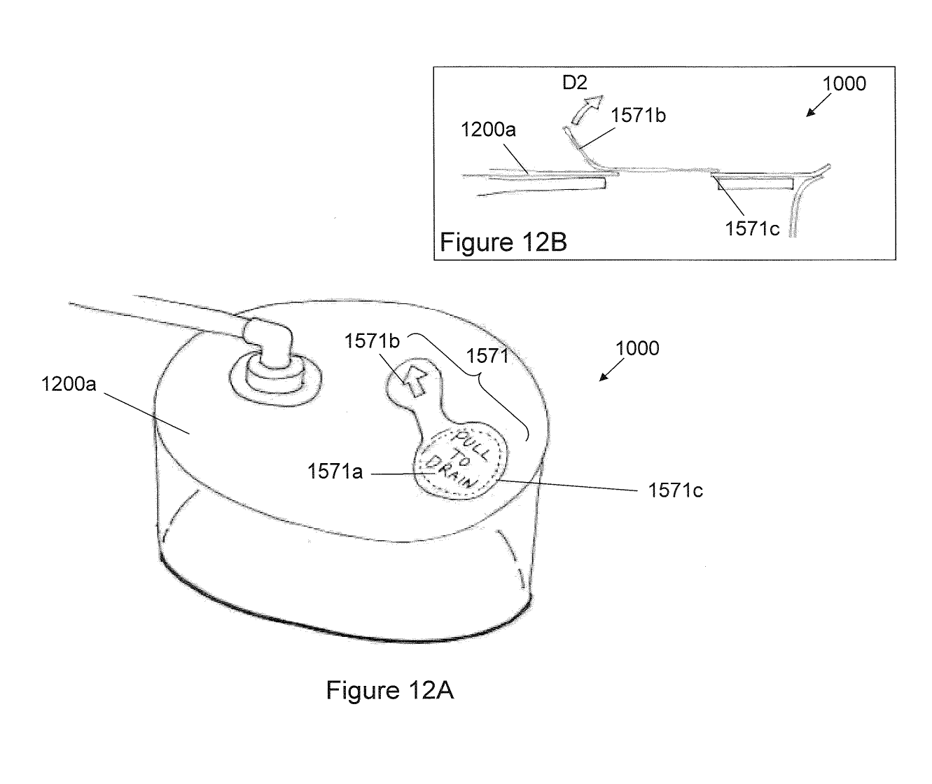

FIGS. 12A and 12B show another exemplary drain system 1571 based on using a tab mechanism 1571a to open a hole in the system 1000. After the user lifts the tab 1571b and pulls it along direction D2 shown in FIG. 12B, amongst other things, a hole opens up in system around seam 1571c. Generally, the seam 1571c will form a ring around the tab 1571b, as shown in FIG. 12A. However, any suitable seam 1571c configuration can be used in the context of the present invention. Subsequent to pulling the tab 1571b and opening a hole in the system 1000, the user may pour out the liquid contents of the system 1000 down a drain or in a waster receptacle. Since the user creates a permanent hole in the system 1000, the system 1000 cannot be re-used. Therefore, the exemplary drain system 1571 is a single-use system that may include the associated advantages discussed above.

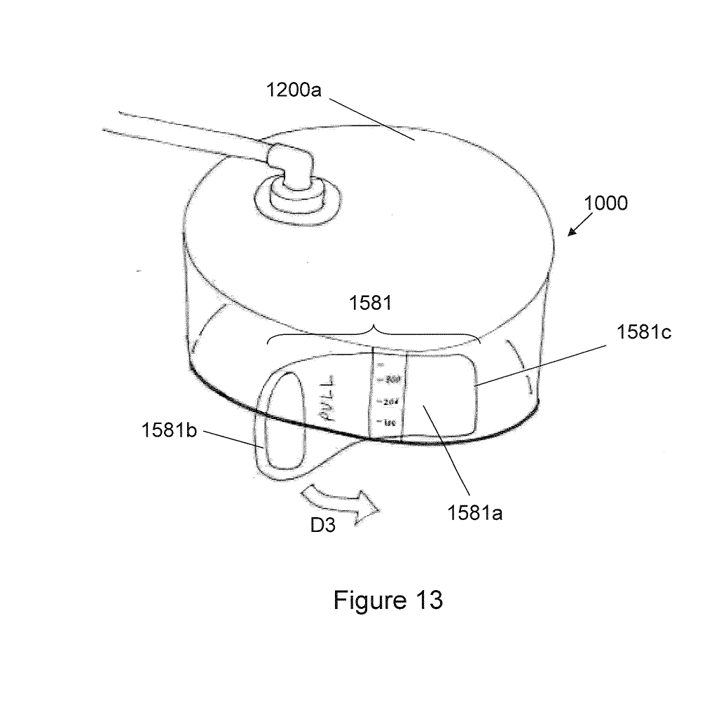

FIG. 13 shows yet another exemplary drain system 1581 based on using a tab mechanism 1581a to open a hole in the system 1000. After the user lifts the tab 1581b and pulls it along direction D3 shown in FIG. 13, amongst other things, a hole opens up in system around seam 1581c. Generally, the seam 1581c will form a ring around the tab, as shown in FIG. 13. However, any suitable seam 1581c configuration can be used in the context of the present invention. Subsequent to pulling the tab 1581b and opening a hole in the system 1000, the user may pour out the liquid contents of the system 1000 down a drain or in a waster receptacle. Since the user creates a permanent hole in the system 1000, the system 1000 cannot be re-used. Therefore, the exemplary drain system 1581 is a single-use system that may include the associated advantages discussed above.

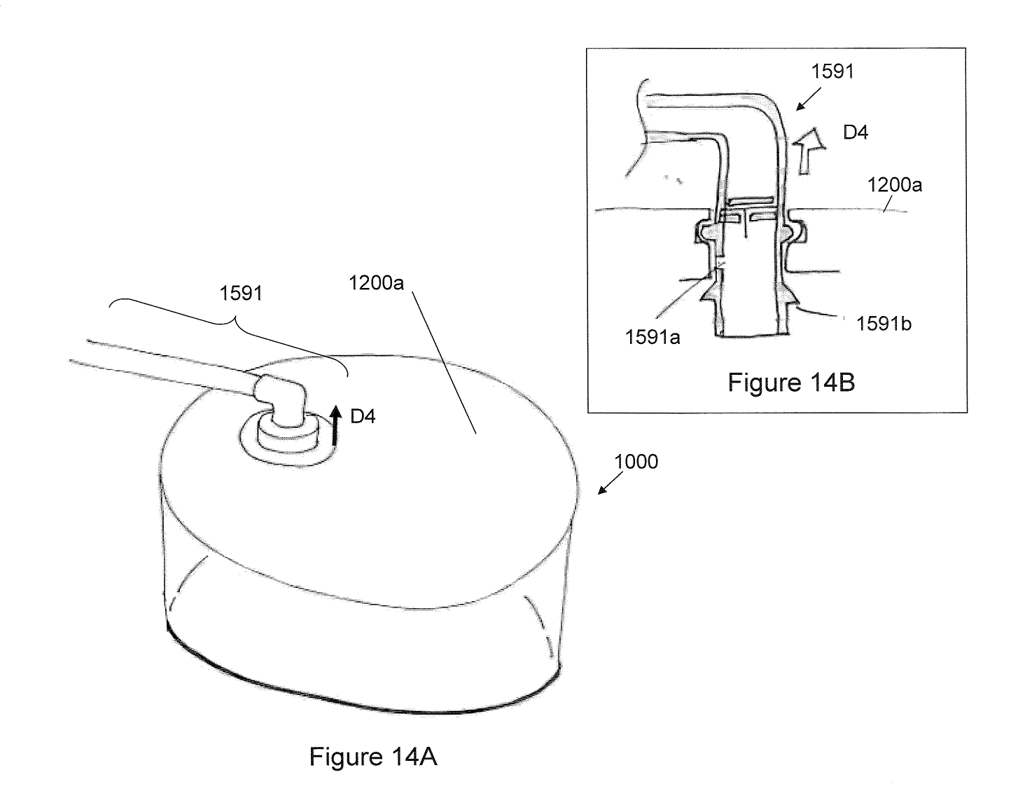

FIGS. 14A and 14B show another exemplary drain system based on a single-use nozzle 1591 to evacuate liquid from the system 1000. The user pulls the single-use nozzle 1591 in direction D4 so that the port 1591a is exposed beyond the surface of the plate 1200a. Once the port 1591a of the single-use nozzle 1591 has been exposed by the user, the user may then drain liquid in the system 1000 through the port 1591a. As shown in FIG. 14B, the single-use nozzle 1591 includes a one-time removal barb 1591b. The one-time removal barb 1591b prevents the re-insertion of the single-use nozzle 1591 into the system 1000 and, thereby, may prevent the user from re-using the system once it has been evacuated of fluid. Therefore, the exemplary drain system 1591 is a single-use system that may include the associated advantages discussed above.

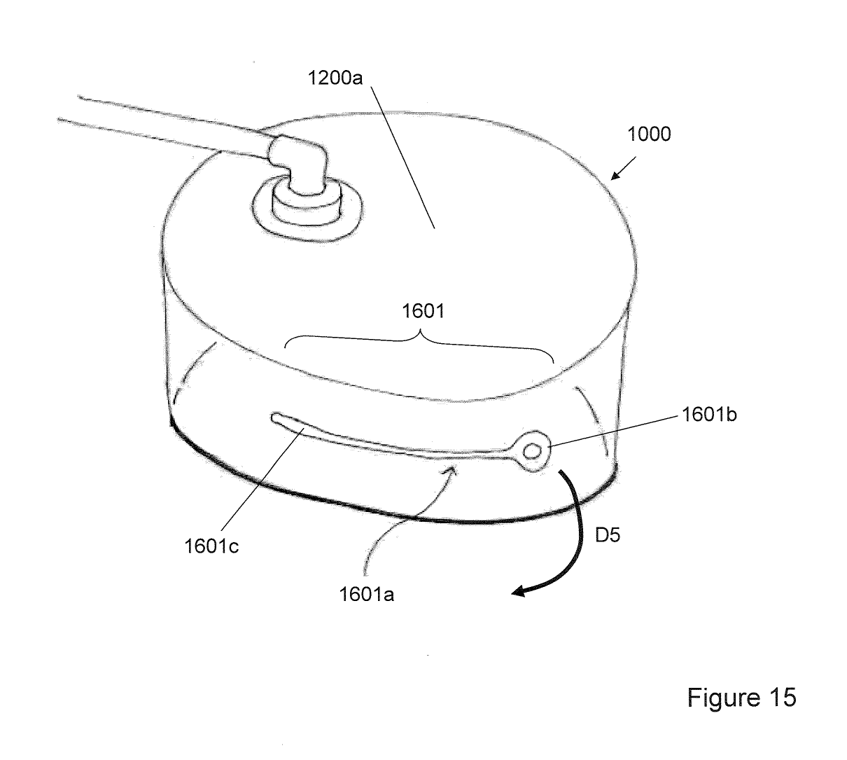

FIG. 15 shows yet another exemplary drain system 1601 based on using a tear strip 1601a to tear open a hole in the system 1000. After the user grabs the handle 1601b and pulls it along direction D5 shown in FIG. 15, amongst other things, a hole opens up in system around seam 1601c. Generally, the tear strip 1601a is an actual strip of fabric or material embedded in the wall of the system 1000 such that pulling the handle 1601b and removing the tear strip 1601a tears the walls of the system along the seam 1601c. Subsequent to pulling the handle 1601b and opening a hole in the system 1000, the user may pour out the liquid contents of the system 1000 down a drain or in a waster receptacle. Since pulling the tear strip 1601a pierces or tears the walls of the system 1000, the system 1000 cannot be re-used. Therefore, the exemplary drain system 1601 is a single-use system that may include the associated advantages discussed above.

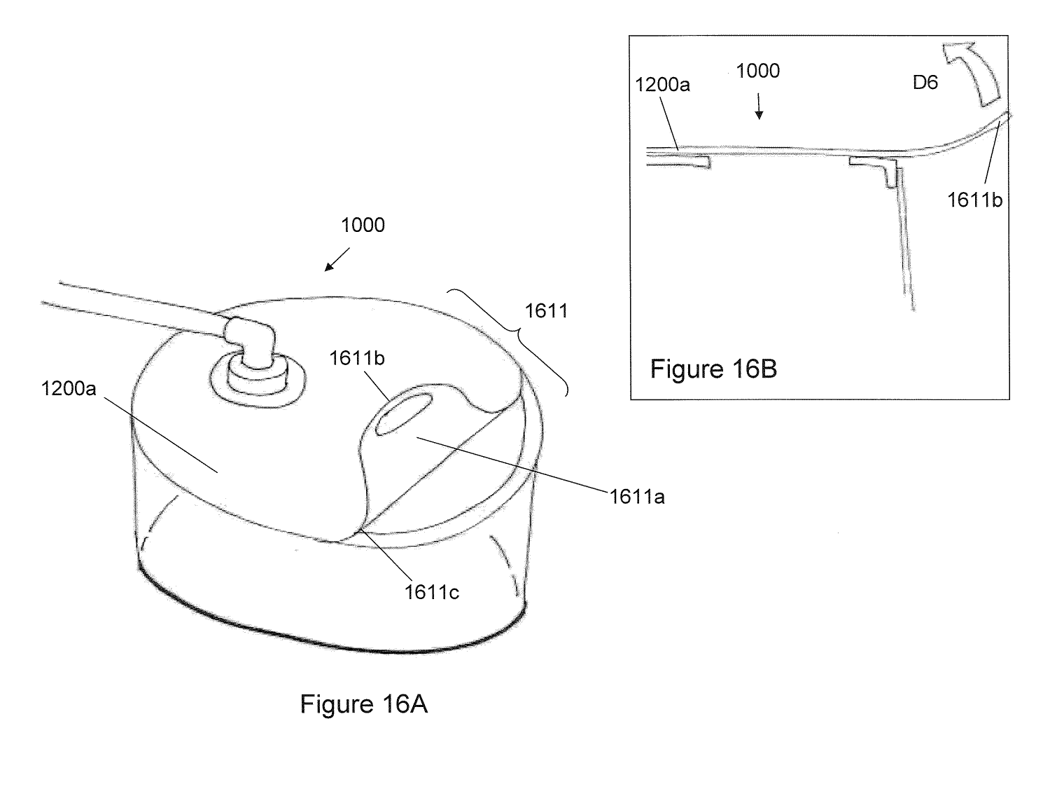

FIGS. 16A and 16B show another exemplary drain system 1611 based on using a cover mechanism 1611a to open a hole in the system 1000. After the user lifts the tab 1611b and pulls it along direction D6 shown in FIG. 16B, amongst other things, a hole opens up in system around seam 1611c. Generally, the seam 1611c will form a ring around cover mechanism 1611a, as shown in FIG. 16A. However, any suitable configuration can be used in the context of the present invention. Subsequent to pulling the tab 1611b and opening a hole in the system 1000, the user may pour out the liquid contents of the system 1000 down a drain or in a waster receptacle. Since pulling tab 1611b pierces or tears the walls of the system 1000, the system 1000 cannot be re-used. Therefore, the exemplary drain system 1611 is a single-use system that may include the associated advantages discussed above.

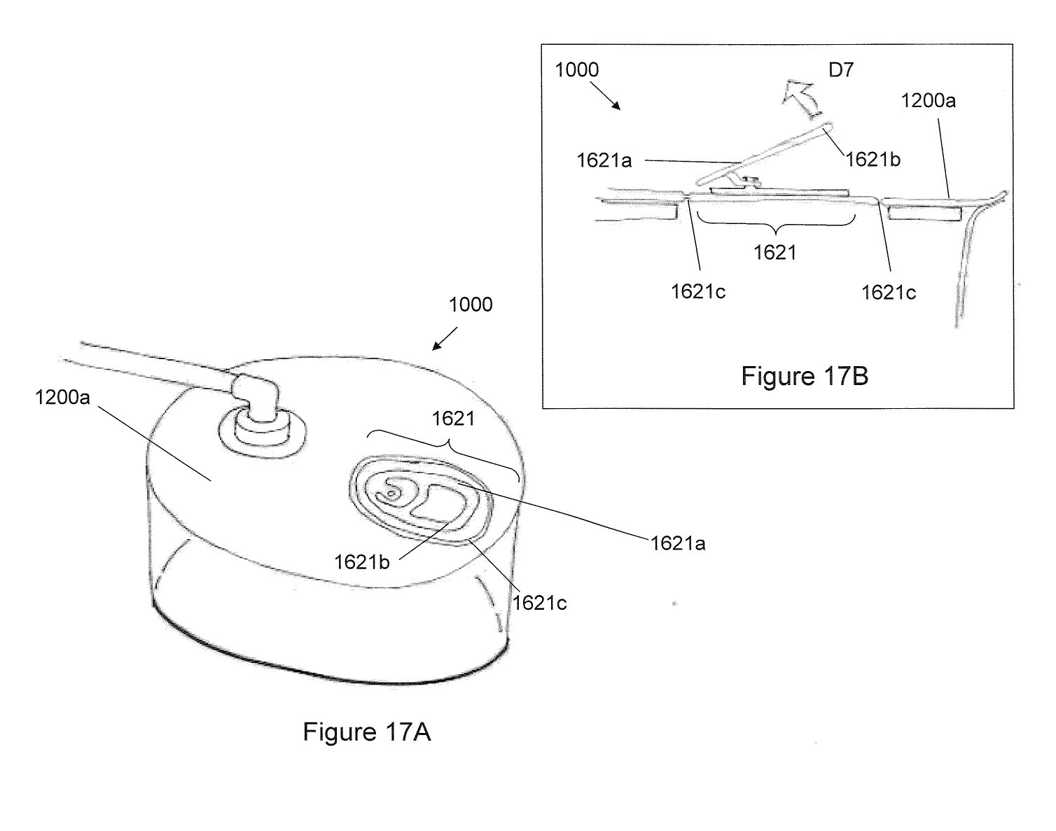

FIGS. 17A and 17B show another exemplary drain system 1621 based on using a tab mechanism 1621a to open a hole in the system 1000. After the user lifts the tab 1621b and pulls it along direction D7 shown in FIG. 17B, amongst other things, a hole opens up in system around seam 1621c. Generally, the seam 1621c will form a ring around the tab, as shown in FIG. 17A. However, any suitable seam 1621c configuration can be used in the context of the present invention. Subsequent to pulling the tab 1621b and opening a hole in the system 1000, the user may pour out the liquid contents of the system 1000 down a drain or in a waster receptacle. Since pulling tab 1621b pierces or tears the walls of the system 1000, the system 1000 cannot be re-used. Therefore, the exemplary drain system 1621 is a single-use system that may include the associated advantages discussed above.

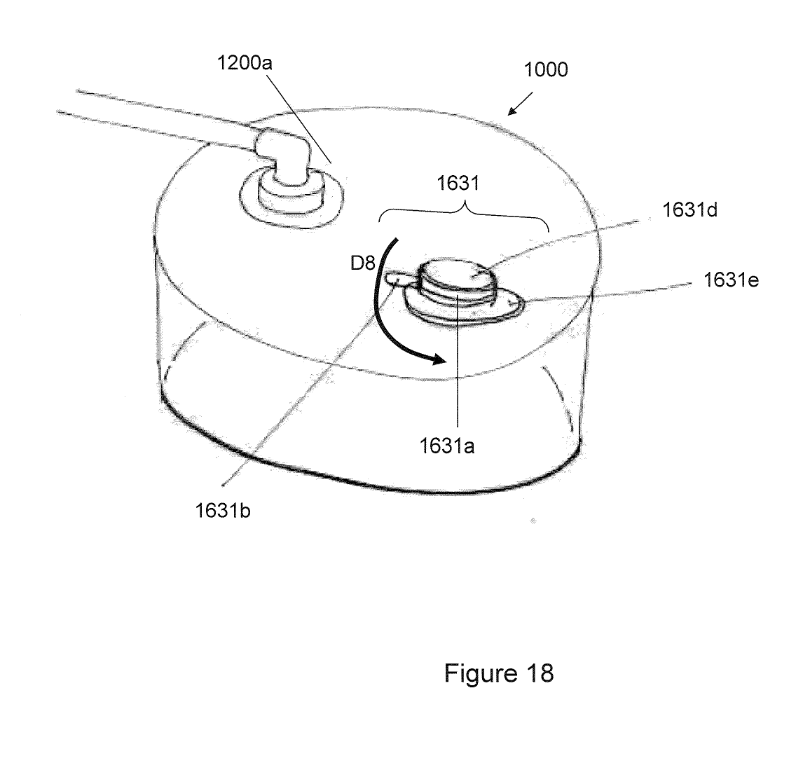

FIG. 18 shows yet another exemplary drain system 1631 based on using a tear strip 1631a. However, the tear strip 1631a differs from that shown in FIG. 15 because, instead of directly tearing a wall of the system 1000, the tear strip 1631a separates two sides 1631d and 1631e of the drain system 1631 from one another. After the user grabs the handle 1631b and pulls it along direction D8 shown in FIG. 18, amongst other things, the two sides 1631d and 1631e of the drain system 1601 separate from one another, opening up a hole to the interior of the system 1000. The side 1631d may be a cap or lid similar to the caps of plastic milk jugs, for example. Generally, the tear strip 1631a is an actual strip of material connecting to the two sides 1631d and 1631e of the drain system 1631 such that pulling the handle 1631b to remove the tear strip 1601a physically separates the two sides 1631d and 1631e of the drain system 1601 from one another. Subsequent to pulling the handle 1631b and opening a hole in the system 1000, the user may pour out the liquid contents of the system 1000 down a drain or in a waster receptacle. Since pulling the tear strip 1631a opens a permanent hole in the system 1000, the system 1000 cannot be re-used. Therefore, the exemplary drain system 1631 is a single-use system that may include the associated advantages discussed above.

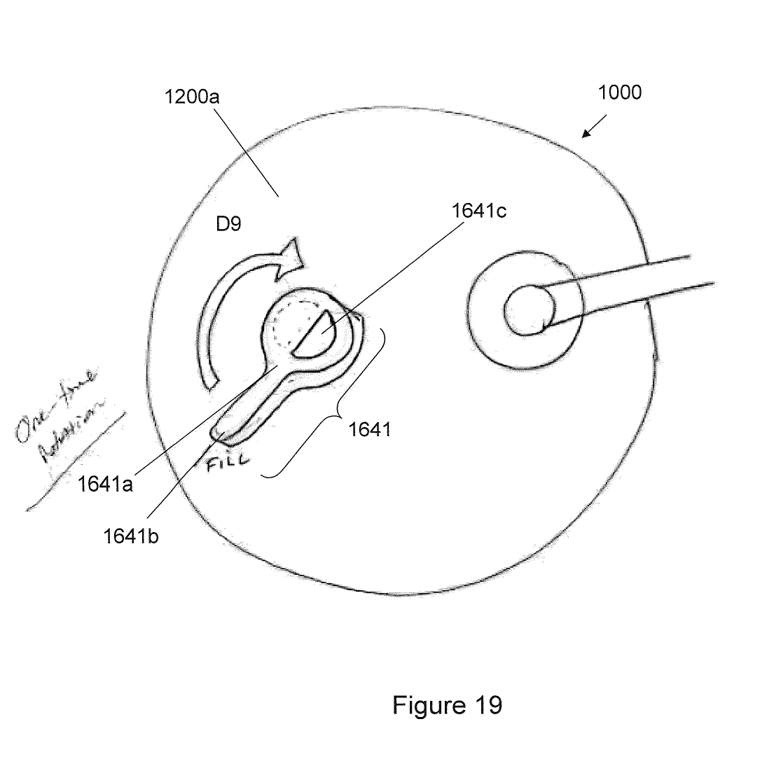

FIG. 19 shows yet another exemplary drain system 1641 based on valve 1641a. Generally, the valve 1641a can be any valve suitable for opening up fluid communication with the interior of the system 1000. The valve 1641a has a handle 1641b and a valve hole 1641c. In the position shown in FIG. 19, i.e., when the handle 1641b is in the “Fill” position, the valve hole 1641c is not exposed to the fluid inside the system 1000. This creates a seal around the valve 1641a such that the system 1000 may be filled with fluid in the manner described above. After the user grabs the handle 1641b and pulls it along direction D9 shown in FIG. 19, amongst other things, the valve hole 1641c is placed in fluid communication with the interior of the system 1000. This allows the system 1000 to be emptied of fluid through the valve hole 1641c. The valve 1641a can be configured such that the valve 1641a cannot be closed or returned to the “Fill” position once opened, thus creating a permanent hole in the system 1000. The valve 1641a may further be configured such that once it is opened to allow fluid to flow through the valve hole 1641c it becomes locked into position. Therefore, the system 1000 cannot be re-used. Therefore, the exemplary drain system 1601 is a single-use system that may include the associated advantages discussed above.

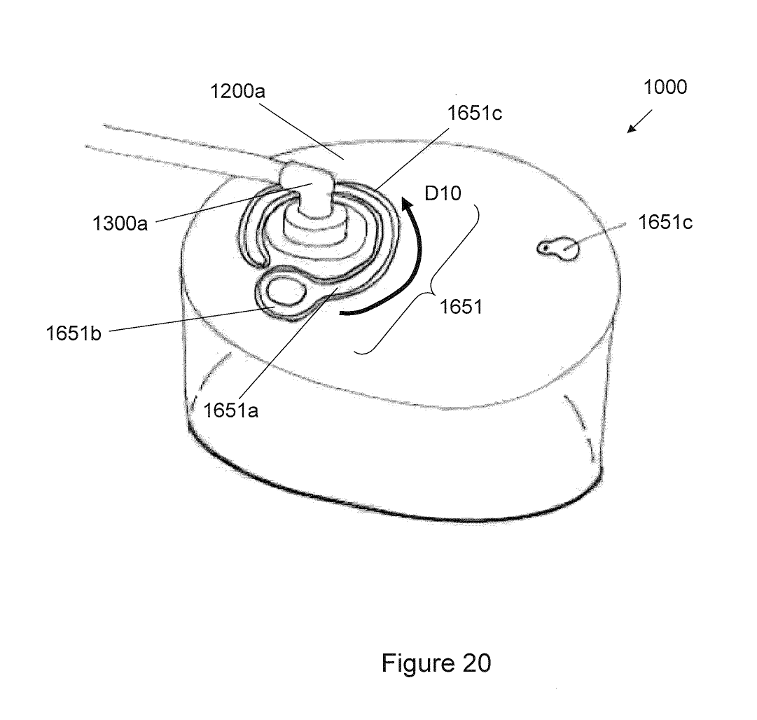

FIG. 20 shows yet another exemplary drain system 1651 based on using a tear strip 1651a. However, the tear strip 1651a differs from that shown in FIG. 15 because, instead of directly tearing a wall of the system 1000, the tear strip 1651a tears a hole around the elbow connector 1300a. After the user grabs the handle 1651b and pulls it along direction D10 shown in FIG. 20, amongst other things, the tear strip 1651a tears the portion of the system 1000 around the elbow connector 1300a along the seam 1651c, opening up a hole to the interior of the system 1000. Generally, the tear strip 1651a is an actual strip of fabric or material embedded in the plate 1200a such that pulling the handle 1651b and removing the tear strip 1651a tears the plate 1200a along the seam 1651c. Subsequent to pulling the handle 1651b and opening a hole in the system 1000, the user may pour out the liquid contents of the system 1000 down a drain or in a waster receptacle. As shown in FIG. 20, the exemplary drain system 1651 may also include an air hole 1651d that may assist in draining. Since pulling the tear strip 1651a opens a permanent hole in the system 1000, the system 1000 cannot be re-used. Therefore, the exemplary drain system 1651 is a single-use system that may include the associated advantages discussed above.

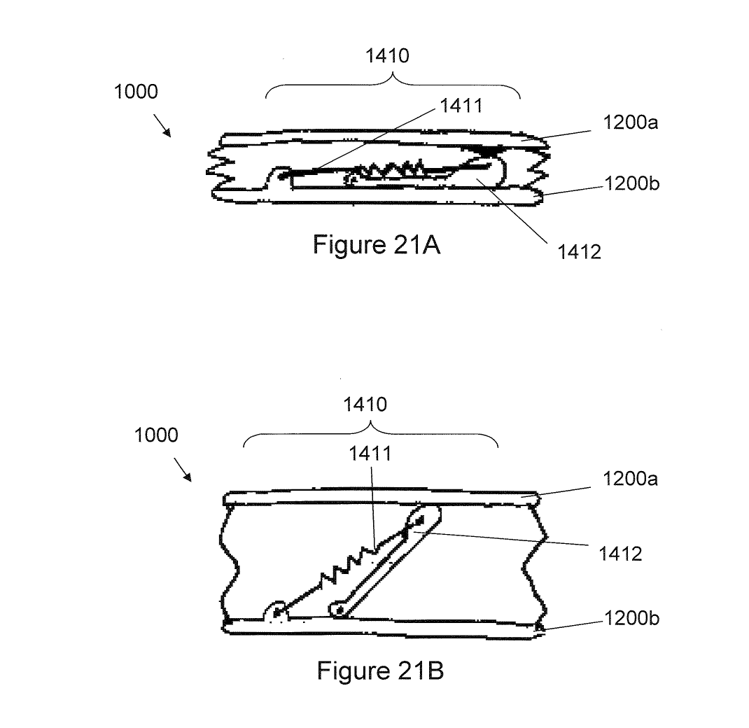

FIGS. 21A and 21B show another exemplary restoring force mechanism 1410 for use with the system 1000. As shown in FIGS. 21A and 21B, the mechanism 1410 may include a spring 1411 or other restoring force member that is attached to a cam 1412. The cam 1412 can provide mechanical advantage to the spring 1411 when the system 1000 is in the extended position shown in FIG. 21B. More specifically, the cam 1412 may allow stronger springs 1411 to be used in the system which may provide an increased force applied to the plates 1200a and 1200b. Although shown in use with only a single spring 1411 or restoring force member in FIGS. 21A and 21B, it is to be understood that the cam 1412 may be used with any suitable number of springs 1411 or restoring force members. Similarly, it is possible to use any suitable number of mechanisms 1410 in a single system 1000 or to use mechanism 1410 with restoring force providing members that are directly attached to the plates 1200a and 1200b as shown in FIG. 1A. It will be appreciated by one skilled in the art that a number of other configurations utilizing these and other components discussed herein are possible within the scope of the invention.

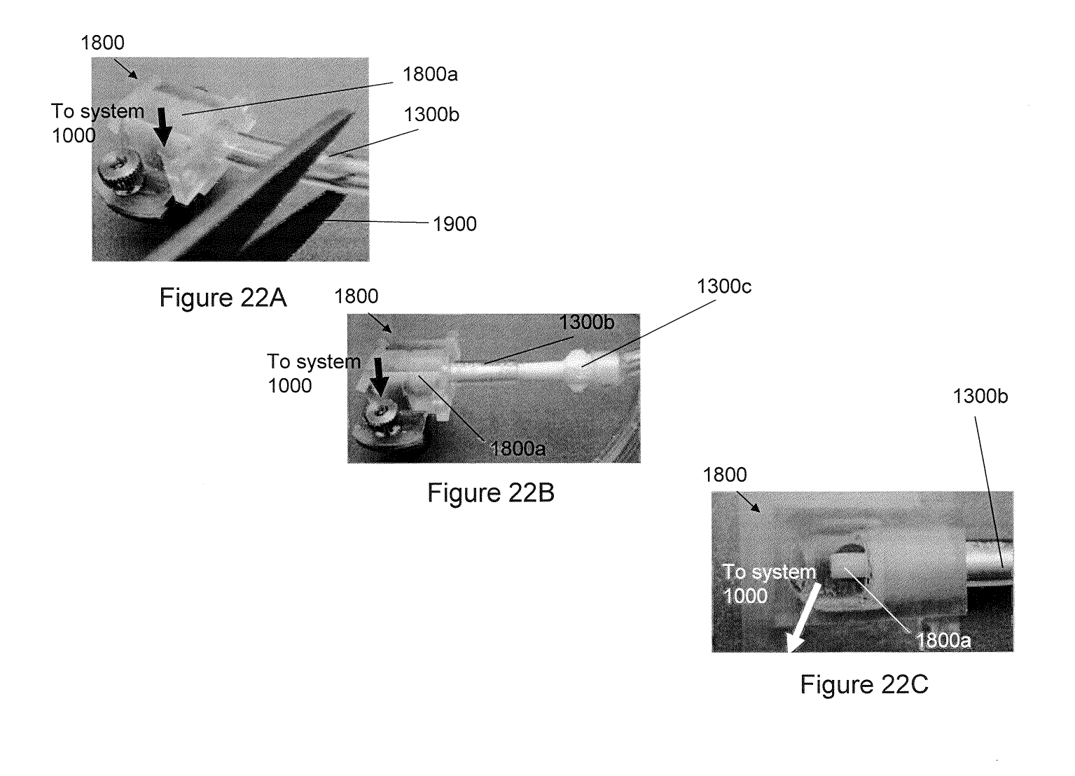

FIGS. 22A-22C show a valve mechanism 1800 that may also be used in accordance with aspects of the present invention. FIGS. 22A and 22B show a top view of user manipulation of the valve mechanism 1800 and FIG. 22C shows a bottom view of the valve mechanism 1800. The valve mechanism 1800 may, for example, be used in place or in conjunction with the cap and drain 1500, or any of the other draining mechanisms discussed herein. As shown in FIGS. 22A-22C, the valve mechanism 1800 can be connected to the hose or intake line 1300b. Inside the valve mechanism 1800, there can be a one-way valve 1800a that, while the system 1000 is being filled with fluid drained from the patient, allows fluid to flow from the hose or intake line 1300b, through the valve mechanism 1800 to the system 1000. The one-way valve 1800a is shown in more detail from the bottom in FIG. 22C. The mode in which fluid flows into the system 1000 may be described as “fill mode” since, in this mode, the valve mechanism 1800 acts to fill the system 1000 with fluid. The mode in which the valve mechanism 1800 is used to drain the system 1000 may be called “drain mode.”

User conversion of the valve mechanism 1800 from fill mode to drain mode is shown in FIGS. 22A and 22B. As shown in FIG. 2A, the hose or intake line 1300b can be cut by the user using scissors 1900, blades, clippers or other cutting tools. Alternatively, the hose or intake line 1300b may have a perforated periphery or a weakened periphery such that the user simply snaps or tears the hose or intake line 1300b without the assistance of tools. Once the hose or intake line 1300b is cut, the user then may insert an access dilator 1300c into the valve mechanism, as shown in FIG. 22B. Insertion of the access dilator 1300c opens the one-way valve 1800a so that it will allow fluid from the system 1000 to flow out through the valve mechanism 1800 and the access dilator 1300c so that it may be discarded. For example, the access dilator 1300c may have a conical shape, as shown in FIG. 22B, such that its insertion mechanically pries open the one-way valve 1800a. Subsequently, the access dilator 1300c acts as a spout for draining fluid (as sown in FIG. 22B) if the interior of the access dilator 1300c is hollow or contains a fluid passageway. Alternatively, the access dilator 1300c may simply be removed, having permanently pried open the one-way valve 1800a such that the system 1000 may now be emptied through the one-way valve 1800a. If the user cuts the hose or intake line 1300b in the manner shown in FIG. 22A, the system 1000 cannot be re-used. Therefore, the exemplary drain system 1651 is a single-use system that may include the associated advantages discussed above.

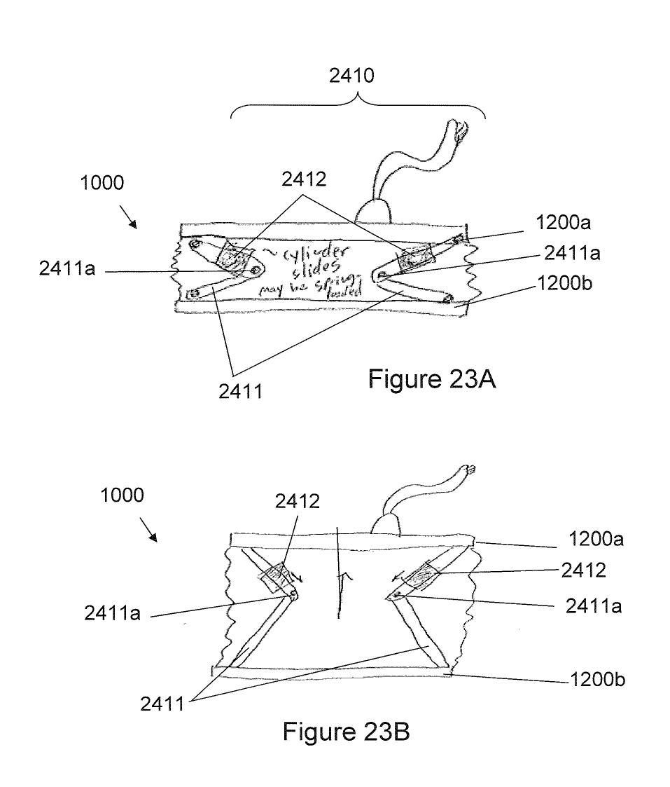

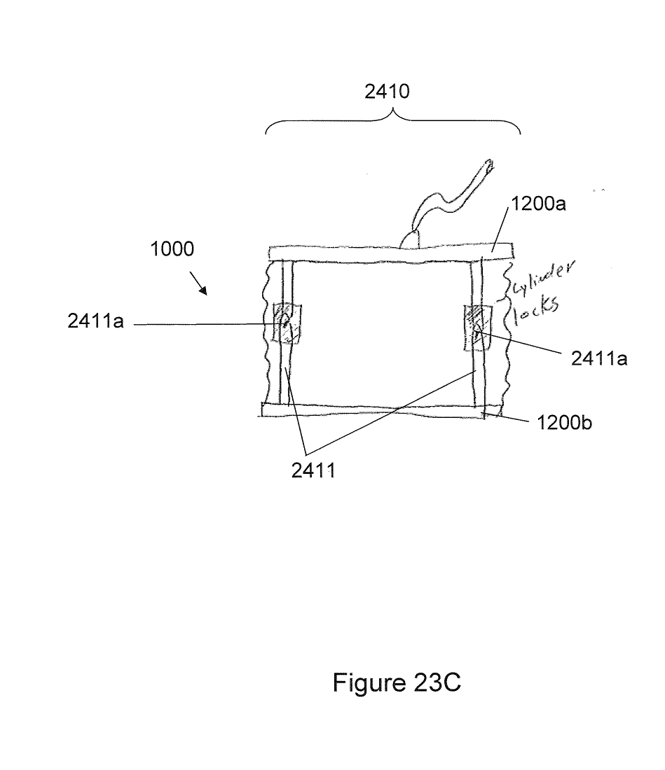

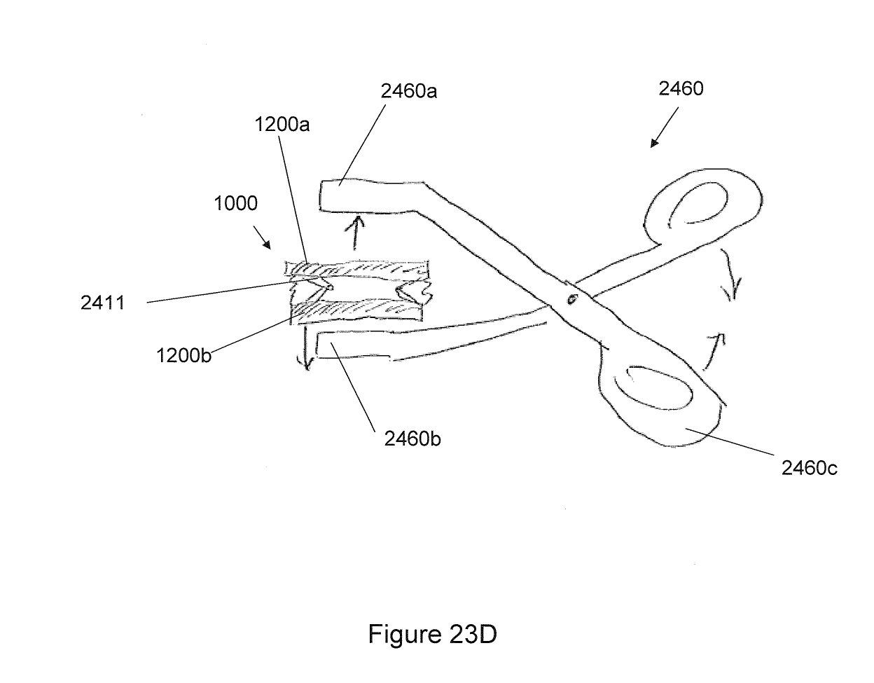

FIGS. 23A-23D show another exemplary restoring force mechanism 2410 for use with the system 1000. As shown in FIGS. 23A-23C, the mechanism 2410 may include a hinges 2411 or other support members that include cylinders 2412. FIG. 23A shows the exemplary restoring force mechanism 2410 in compressed mode when the hinges 2411 are bent at the pivot point 2411a and the cylinders 2412 are positioned so that they do not cover the principal pivot point 2411a. As shown in FIG. 23B, the hinges 2411 can be extended by rotating about the pivot point 2411a. Once the hinges 2411 are in the straightened position shown in FIG. 23C, the cylinders 2412 can be slid over the pivot points 2411a. Sliding the cylinders 2412 over the pivot points 2411a can, for example, fix the hinges 2411 and prevent them from bending at the pivot points 2411a. Fixing the hinges in this way, may make it possible for the system 1000 to pull a vacuum for the collection and drainage of fluid. The cylinders 2412 may be designed such that they cannot be removed from the pivot points 2411a once placed on them. In this and other ways, the exemplary restoring force mechanism 2410 may be a single-use system with all of the advantages described herein.

It is possible to use any suitable number of mechanisms 2410 in a single system 1000 or to use mechanism 2410 with hinges 2411 that are directly attached to the plates 1200a and 1200b or in another suitable configuration. It will be appreciated by one skilled in the art that a number of other configurations utilizing these and other components discussed herein are possible within the scope of the invention. It should be appreciated that the above-described relationship between the cylinders 2412 and hinges 2411 can be accomplished using other suitable configurations. For example, the cylinders 2412 may be replaced by a screw or other fastening mechanism. Any of the fastening mechanisms discussed herein may be suitably used in conjunction with the hinges 2411.

FIG. 23D shows an exemplary reusable external device 2460 that may be used to “charge” system 1000 by compressing it. For example, the user may grasp handle 2460c and squeeze in a similar manner to using a pair of scissors. This may, for example, squeeze the ends 2460a and 2460b such that they press on the plates 1200a and 1200b, as shown in FIG. 23D. Such an action may, for example, lock the hinges 2411 in a certain position that more effectively pulls a vacuum. Moreover, the construction of the hinges 2411 and the device 2460 may allow increased mechanical advantage to the user when compressing the system 1000 to create a vacuum. It is to be understood that the exemplary external device 2460 may be used with any of the variations of the system 1000 and other systems discussed herein.

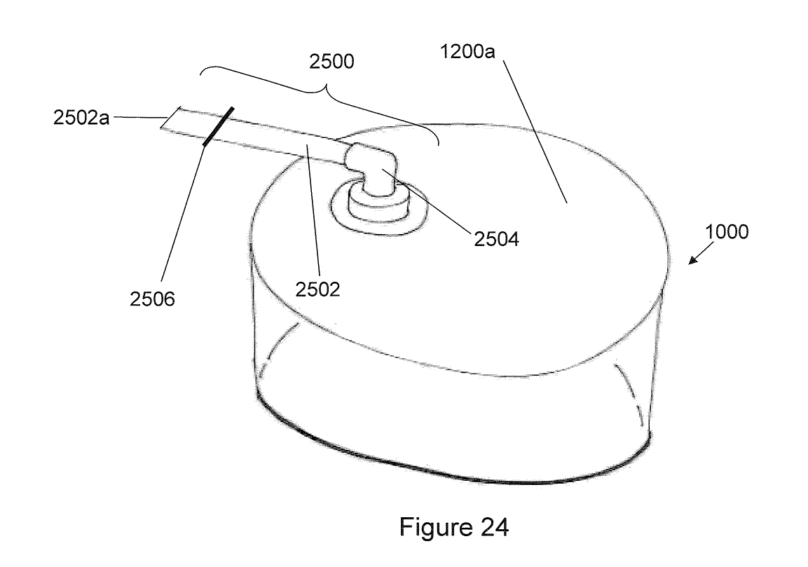

FIG. 24 shows another exemplary drain system 2500 based on a tube 2502 to evacuate liquid from the system 1000. The tube 2502 may be straight, as shown, or it may occur in other suitable configurations. For example, the tube 2502 may be disposed as a coil such that the user unrolls the coil before it is used. The tube 2502 is generally connected to an elbow or other type of joint 2504 that serves as a fluid conduit between the tube 2502 and the system 1000. The elbow or other type of joint 2504 may be any suitable structure that allows fluid communication between the system 1000 and the tube 2502. The tube 2502 may be clamped shut using a clamp 2506, as shown in FIG. 24. Clamping the tube 2502 with the clamp 2506 may, for example, prevent fluid from the system 1000 from exiting through the tube end 2502a. Releasing the clamp 2506, on the other hand, may prevent fluid to flow from the system 1000 through the tube end 2502a for draining. The clamp 2506 may be configured so that it may only be released a single time. Therefore, the exemplary drain system 2500 can be used as a single-use system that may include the associated advantages discussed above.

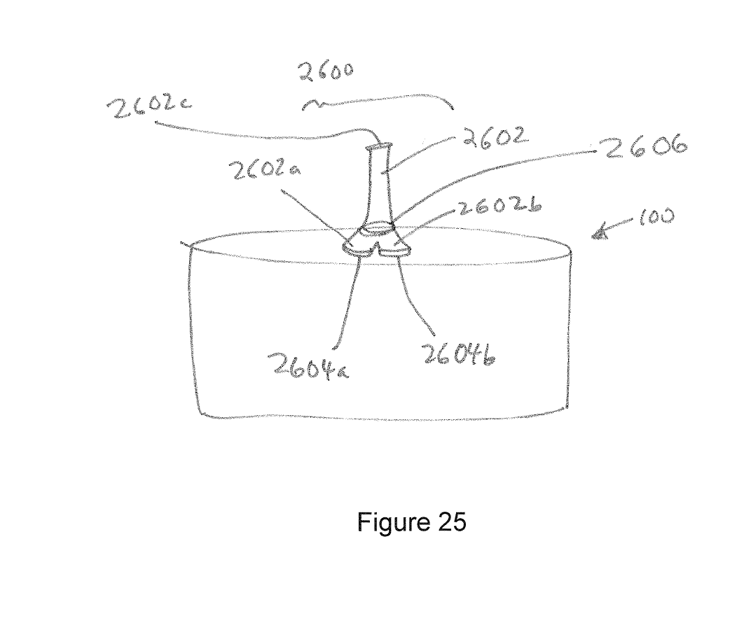

FIG. 25 shows an exemplary Y-valve drainage system 2600 that may be used according to aspects of the present invention. The Y-valve drainage system 2600 includes a Y-valve 2602 with two or more ends (e.g., 2602a and 2602b shown in FIG. 25). The two or more ends 2602a and 2602b may be further connected to valves 2604a and 2604b. In certain configurations, the valves 2604a and 2604b may be oppositely configured, one-way valves. For example, valve 2604a may be configured to allow fluid to flow only from the system 1000 to the Y-valve 2602, but not from the Y-valve 2602 to the system 1000. Correspondingly, valve 2604b may be configured to allow fluid to flow only from the Y-valve 2602 to the system 1000, but not from the system 1000 to the Y-valve 2602. The system 1000 may then be drained through valve 2604a and filled through valve 2604b. The Y-valve drainage system 2600 may further include a switch 2606 that switches the fluid connection between the end 2602c of the Y-valve 2602 and the valves 2604a and 2604b. For example, the switch 2606 may be set such that there is fluid communication between the valve 2604a and end 2602c so that the system 1000 can be drained. During draining, the switch 2606 would cut off fluid communication between the end 2602c and the valve 2604b. Alternatively, the switch 2606 may be set such that there is fluid communication between the valve 2604b and end 2602c so that the system 1000 can be filled. During filling, the switch 2606 would cut off fluid communication between the end 2602c and the valve 2604a. Substantially the reverse valve configurations, as well as other suitable configurations, are also possible.

Although the invention has been described with reference to various aspects of the present invention and examples with respect to a corporal drainage application, it is within the scope and spirit of the invention to incorporate or be used in conjunction with any suitable medical or other devices. Further, while the invention has been describe with reference to medical or body fluid extraction, the invention may be used with other applications, depending on circumstances in which the invention is used. Thus, it should be understood that numerous and various modifications may be made without departing from the spirit of the invention.

What is claimed is:

1. A corporal drainage system for draining fluid from a patient including;
two plates positioned opposite each other;
at least one restoring force providing member positioned between the plates;
a mechanism for securing the plates in a compressed mode wherein the at least one restoring force providing member is compressed;
a mechanism for releasing the two plates in an extended mode wherein the at least one restoring force providing member is extended;
a seal between the two plates and an intake port, wherein such that when the plates are in the extended mode, a vacuum is exerted on the intake port; and
a conduit connecting the intake port to the patient, wherein the vacuum exerted on the intake port draws fluid from the patient.

2. The rapid corporal drainage system of claim 1, wherein the restoring force providing member has a characteristic length which is a length of the restoring force providing member when not being acted on by an outside force, and
wherein in the extended mode, the length of the restoring force providing member is less than the characteristic length.

3. The rapid corporal drainage system of claim 1, wherein a compressed mode ratio defined as a ratio of a distance between the plates in the compressed mode divided by a distance between the plates in the extended mode is 0.40 or less.

4. The rapid corporal drainage system of claim 3, wherein the compressed mode ratio is 0.25 or less.

5. The rapid corporal drainage system of claim 1, wherein the mechanism for securing the plates to one another in the compressed mode includes straps that wrap around the plates.

6. The rapid corporal drainage system of claim 5, wherein the straps include a hook and loop mechanism for securing the plates to one another in the compressed mode.

7. The rapid corporal drainage system of claim 5, wherein the mechanism for releasing the two plates includes a handle for pulling apart a hook portion and a loop portion of the straps.

8. The rapid corporal drainage system of claim 5, wherein the plates include ribs for accommodating the straps.

9. The rapid corporal drainage system of claim 5, wherein the plates include scalloping for accommodating the straps.

10. The rapid corporal drainage system of claim 5, wherein the straps include snap fasteners for securing the plates to one another in the compressed mode.

11. The rapid corporal drainage system of claim 1, including a single-use drain mechanism.

12. The rapid corporal drainage system of claim 11, wherein the single-use drain mechanism includes a tear strip.

13. The rapid corporal drainage system of claim 11, wherein the single-use drain mechanism includes a tab.

14. The rapid corporal drainage system of claim 11, wherein the single-use drain mechanism includes a puncture tool for puncturing a portion of the system and draining the system through the punctured portion of the system.

15. The rapid corporal drainage system of claim 11, wherein the single-use drain mechanism includes a one-way valve and an access dilator, and wherein the access dilator opens the one-way valve to remove the fluid through the one-way valve.

16. The rapid corporal drainage system of claim 1, wherein the mechanism for releasing the two plates in an extended mode includes a handle portion that the user may pull.

17. The rapid corporal drainage system of claim 1, wherein the mechanism for securing the plates in a compressed mode includes binding straps together and the mechanism for releasing the two plates in an extended mode is activated by at least one of: turning a release mechanism, releasing springs or buckles, unfastening a pin, unzipping a zipper, unclipping a clip or undoing a tab, clasp or loop.

18. The rapid corporal drainage system of claim 1, further including a drain mechanism that includes a tab or tear strip that can be pulled to tear a hole in the side of the rapid corporal drainage system.

19. The rapid corporal drainage system of claim 1, further including a drain mechanism that includes a single-use nozzle that may be pulled by the user to expose a port for draining the rapid corporal drainage system.

20. The rapid corporal drainage system of claim 1, further including a drain mechanism that includes a cover mechanism that can be opened by pulling a tab attached to the cover mechanism.

21. The rapid corporal drainage system of claim 20, wherein the tab is configured such that pulling the tab presses on the cover mechanism to create an opening.

22. The rapid corporal drainage system of claim 1, further including a drain mechanism that includes a tear strip configured such that pulling the tear strip separates two sides of the drain mechanism from one another.

23. The rapid corporal drainage system of claim 1, further including a drain mechanism that includes a single-use valve with a handle and a valve hole.

24. The rapid corporal drainage system of claim 23, wherein the valve is twisted to open the valve hole and wherein, once the valve is twisted to open the valve hole, the valve is locked into place.

25. The rapid corporal drainage system of claim 1, further including a drain mechanism that includes a tear strip configured to tear a hole around an elbow connector.

26. The rapid corporal drainage system of claim 1, further including a drain mechanism that includes a blocking material that prevents unwanted fluid spray during draining.

27. The rapid corporal drainage system of claim 1, further including a drain mechanism that includes a membrane under a tear-away cap that prevents reflux from occurring when the cap is removed.

28. The rapid corporal drainage system of claim 1, further including a drain mechanism that includes a piece of tubing that is configured to be unclamped and unrolled to drain the rapid corporal drainage system.

29. The rapid corporal drainage system of claim 1, further including a drain mechanism that includes a Y connector that accesses two one-way valves, wherein one of the one-way valves is configured to allow draining of the rapid corporal drainage system.

30. The rapid corporal drainage system of claim 1, wherein the restoring force providing member includes a spring attached to a cam such that the cam provides mechanical advantage to the spring.
